(19) United States
(12) Patent Application Publication
(10) Pub. No.: US 2021/0038742 A1
(43) Pub. Date: Feb. 11, 2021

(54) CONJUGATION OF TLR7 AGONIST TO NANO-MATERIALS ENHANCES THE AGONISTIC ACTIVITY

(71) Applicants: Ching-Hsin Huang, La Jolla, CA (US); Natalie Mendez, La Jolla, CA (US); James Wang, La Jolla, CA (US); Tomoko Hayashi, San Diego, CA (US); Joi Weeks, San Diego, CA (US); Oscar Hernandez Echeagaray, San Diego, CA (US); Andrew Kummel, San Diego, CA (US); William C. Trogler, Del Mar, CA (US); Natalie A. Gude, San Diego, CA (US)

(72) Inventors: Ching-Hsin Huang, La Jolla, CA (US); Natalie Mendez, La Jolla, CA (US); James Wang, La Jolla, CA (US); Tomoko Hayashi, San Diego, CA (US); Joi Weeks, San Diego, CA (US); Oscar Hernandez Echeagaray, San Diego, CA (US); Andrew Kummel, San Diego, CA (US); William C. Trogler, Del Mar, CA (US); Natalie A. Gude, San Diego, CA (US)

(21) Appl. No.: 16/906,933

(22) Filed: Jun. 19, 2020

Related U.S. Application Data
(60) Provisional application No. 62/864,249, filed on Jun. 20, 2019.

(51) Int. Cl. A61K 47/69; A61K 31/522; A61K 31/437; A61P 35/04

(52) U.S. Cl. CPC A61K 47/6923; A61K 31/522; A61K 9/0019; A61P 35/04; A61K 31/437

(57) ABSTRACT

A composition comprising silica shells conjugated to a TLR7 agonist and methods of using the composition are provided.

### CROSS-REFERENCE TO RELATED APPLICATIONS

This application claims the benefit of the filing date of U.S. application No. 62/864,294, filed on Jun. 20, 2019, the disclosure of which is incorporated by reference herein.

### BACKGROUND

Resiquimod and imiquimod have several mechanisms of action, being both an agonist for toll-like receptor (TLR) 7 and 8. Resiquimod and imiquimod are used as a topical cream (e.g., Aldera) to avoid side effects (Mark et al., 2007; Engel et al., 2011; Meyer et al., 2013). 1 Side effects include local inflammatory reactions, such as burning sensation, skin redness, dry skin, or even more serious side effects such as the cytokine syndrome Soria et al., 2003). 1V209, imiquimod, SM360320 are potent unconjugated TLR7 or TLR7/8 agonists that can activate the NF-kB to induce IL-6 and IL-12 production in antigen presenting cells (macrophages, dendritic cells) (Chan et al., 2009; Dockrell, 2001; Kawai et al., 2015; Shinchi et al., 2015). However, since these TLR7 or TLR 7/8 agonists are small molecular weight molecules, these compounds showed rapid pharmacokinetics in vivo (Engel et al., 2011; Milling et al., 2017; Al-Abd et al., 2010).

### SUMMARY

Mono- or dual-checkpoint inhibitors for immunotherapy have changed the paradigm of cancer care. However, only a minority of patients is responsive to such treatment. As described below, a nanoparticle-based TLR7 agonist, e.g., (1V209, (4-{[6-amino-2-(2-methoxyethoxy)-8-oxo-7H-purin-9(8H)-yl]methyl}benzoic acid), was employed to improve immune stimulatory effects induced by activation of TLR7 signaling pathway. TLR7 agonists conjugated onto silica nanoparticles enhanced the immune cytokine secretion and lengthened the drug localization compared to unconjugated agonists. When treated on CT26 murine syngeneic colon cancer, nanoparticle conjugated TLR7 agonists increased T cell infiltration into tumor and up-regulated the expression level of IFN-γ gene. The toxicity reports indicated that conjugated TLR7 agonist is (to silica nanoparticles) were a safe agent at the effective dose. The conjugated TLR7 agonists (to silica nanoparticles) were combined with checkpoint inhibitors anti-PD-1 and anti-CTLA4 and a significant improvement of the immune cell migration and the excellent treatment efficacy on colon cancers were observed. The data has shown that such a combination can more effectively inhibit tumor progress, induce full remission on both a directly treated tumor and a contralateral untreated tumor, and establish a long-term tumor specific memory immune function.

In particular, synthetic TLR ligands were conjugated onto nanomaterials to form an immunotherapy drug complex that augments the immune-stimulatory activity of TLR ligands and increases the retention rate. Silica nanoshells were engineered with a small molecule toll-like receptor 7 (TLR7) agonist, 1V209, and those molecules displayed enhanced adjuvant activities in vitro and in vivo. The TLR7 agonist-silica nanoshell conjugates can enhance the production of proinflammatory cytokines (e.g., IL-6, IL-12 etc.) in bone marrow derived dendritic cells (BMDCs) and promote the expression of co-stimulatory molecules (e.g., CD80, CD40, and/or CD86) on dendritic cells. When administered intratumorally in mice, the TLR7 agonist-silica nanoshell conjugates induced transient higher levels of proinflammatory and type-1 interferon cytokines in serum, compared to the unconjugated form of TLR7 agonists and silica nanoshells. When the TLR7 agonist-silica nanoshell conjugates were used as the cancer vaccine adjuvants, TLR7 agonist-nanosilica shells enhanced protein/antigen specific humoral and cellular immune responses to a much greater extent than unconjugated small molecule TLR7 agonists. When the TLR7 agonist-silica nano shell conjugates were used as a therapeutic agent for cancer treatment, the conjugates showed a higher T cell infiltration in tumor microenvironment compared to unconjugated TLR7 agonists or silica alone. By combining TLR7 agonist silica nanoshell conjugates with checkpoint inhibitor and ultrasound-guided histotripsy, not only can the injected tumor be induced into remission, but an uninjected contralateral tumor can also be induced into remission by induction of tumor specific immune responses.

The disclosure thus provides a composition comprising a plurality of silica nanoshells conjugated to a TLR7 agonist. In one embodiment, the shells have a diameter less than about 200 nm. In one embodiment the shells have a diameter less than about 150 nm. In one embodiment, the shells have a diameter less than about 100 nm. In one embodiment, the shells have a diameter greater than about 20 nm. In one embodiment, the shells have a diameter greater than about 50 nm. In one embodiment, the TLR7 agonist has formula (II). In one embodiment, the TLR 7 agonist comprises 1V209 or SM360320. In one embodiment, the composition further comprises one or more checkpoint inhibitors. In one embodiment, the inhibitor comprises pembrolizumab, nivolumab, atezolizumab, avelumab, durvalumab, or ipilimumab. In one embodiment, one or more checkpoint inhibitors are administered before, concurrently with (e.g., in a composition separate from the TLR7 agonist), or after the TLR7 agonist is administered.

Also provided is a method to inhibit or treat cancer. The method includes administering to a mammal in need thereof an effective amount of the composition. In one embodiment, the mammal is a human. In one embodiment, a chemotherapeutic or anti-cancer antibody, e.g., that is a checkpoint inhibitor, is also administered to the mammal, e.g., pembrolizumab, nivolumab, atezolizumab, avelumab, durvalumab, or ipilimumab is administered to the mammal. In one embodiment, the composition is parenterally administered. In one embodiment, the composition is intravenously administered. In one embodiment, the composition is locally administered. In one embodiment, the composition is intratumorally administered. In one embodiment, the method also includes employing ultrasound guided histotripsy. In one embodiment, the composition is injected into the tumor. In one embodiment, the amount enhances pro-inflammatory cytokines. In one embodiment, the amount enhances pro-co-stimulatory molecule expression in dendritic cells. In one embodiment, the cancer is bladder cancer, prostate cancer, testicular cancer, lung cancer, breast cancer, brain cancer, liver cancer, ovarian cancer, kidney cancer, or pancreatic cancer.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Mechanism of action of TLR7 signaling pathway in antigen-presenting cells (APC) on anti-tumor immunity.

FIGS. 2A-2C. Characterization of TLR7 agonists-silica shell conjugates: Images of transmission electron microscopy of A) 100 nm non-modified silica nanoshells and B) 100 nm silica 1V209 (2-methoxyethoxy-8-oxo-9-(4-carboxy benzyl)adenine)-nanoshell conjugates. No morphological differences were observed. C) Fluorescence spectra of 1V209-SiO₂ nanoshell conjugates.

FIG. 3. Tracking of TLR7 agonists-silica nanoshells in BMDCs reveals late endosome localization. BMDCs were incubated with TLR7 agonists-silica shells (1V209 NS, pink) for up to 6 hours. Live cell dyes for the late endosome/lysosomes (green), nuclei (white) and membrane (blue) were added 5-30 minutes before acquisition. TLR7 agonists-silica shells were trafficked into the cytosol compartment, and the majority of nanoshell conjugates were in the late endosome/lysosome compartments at 6-hour post-treatment.

FIGS. 4A-4E. TLR7-silica shell conjugates enhance in vitro cytokines of TLR7 signaling pathway and demonstrate low toxicity in BMDCs. Mouse BMDCs or human PBMCs were plated and incubated with serially diluted 1V209 nanoshells conjugates at different coating densities (high, medium, or low density conjugates, unconjugated TLR7 ligand 1V209, or silica nanoshells for 18 hours. Control cells were treated with 1 μM resiquimod or 0.5% DMSO as vehicle. IL-6 (A) and IL-12 (B) released in the culture supernatants of mouse BMDCs were measured using ELISA. (C) Cytotoxicity in BMDCs was measured by MTT assay. (D) IL-6 released in the culture supernatants of human PBMCs. (E) IL-6 released in the culture supernatants BMDCs isolated from TLR7 deficient mice.

FIGS. 5A-5C. TLR7 agonist-silica nanoshells conjugates generate high level of IL-1p in BMDCs. 75-2500 nM of TLR7 agonist-silcia shells conjugates were incubated with BMDCs for 24 hours at 37° C., 5% CO₂. IL-1β level in supernatant was determined by ELISA. Mechanism figure reference: He, Y. et. al. (2016).

FIGS. 6A-6B. Immunization study of TLR7 ligands-silica nanoshells conjugates with ovalbumin (OVA) as an antigen. To study whether the conjugation with TLR7 agonist to silica particle augments immune adjuvant effects, BALB/c mice (n=4) were intramuscularly immunized with OVA and TLR7 agonist-silica nanoshells conjugates, mixture of TLR7 ligands and silica nanoshells, silica nanoshells and unconjugated TLR7 ligand (1V209), on days 0 and 20, and sacrificed on day 42. The levels of sera anti-OVA IgG1 antibody IgG2a antibody were determined by ELISA. Splenocytes were stimulated for 5 days at 37° C., 5% CO₂ with OVA and the levels of IFN gamma in the culture supernatants were tested by ELISA.

FIGS. 7A-7H. Plasma cytokine levels with 1V209 and 1V209-silica shells conjugates treatments (1V209-silica nanoshells conjugates are denoted as conj. in the figure).

FIG. 8. Mechanism of action of therapeutic effects of TLR7 ligands conjugated with silica nanoshells with anti-PD-1 antibody and histropsy in colon cancer. Silica shells assisted high intensity focused ultrasound (NS-assisted HIFU) lyses tumor cells and release naïve antigen to stimulate and recruit antigen presenting cells (APC). TLR7 agonists-silica shells conjugates (1V209-NS conjugate) promote APC maturation and induce T cell infiltration into tumor microenvironment. Checkpoint inhibitor (anti-PD 1) re-store T cells' killing capability to destroy tumors.

FIGS. 9A-9C. Localized 1V209 NS treatment prolongs TLR7 signaling, enhances antigen presentation by APCs, and increases CD3⁺ T cell infiltration compared to 1V209 alone. (A) Treated tumors immunolabeled for CD3 (B) Quantification of CD3 cells in treated tumors (C) IFNg transcriptional level is upregulated by 1V209-NS conjugates.

FIGS. 10A-10D. Combination of TLR7 agonists-nanosilica shells, anti-PD1, HIFU can induce systemic anti-tumor immunity. BALB/c (n=4-5/group) were implanted with 10⁶ CT26 colon tumor cells into right and left flasks. HIFU was treated on one site of tumor on day 0 with tumor average size 100 mm³. Mice were intratumorally (i.t.) treated with TLR7 agonists-silica shells (1V209-NS) every other day and i.p. treated with anti-PD1 three times per week. Both sites of tumors were monitored for tumor progression.

FIGS. 11A-11B. Combination therapy with i.t. TLR7 agonists-nanosilica shells conjugates and systemic anti-PD-1 antibody and HIFU increases CD8+ cell infiltration (left panel) and INF-γ gene expression (right panel) in tumors.

FIGS. 12A-12H. Tumor growth regression in mice treated with TLR7 agonists-silica nanoshells conjugates (4 different treatments). Individual growth curves for each treatment group are shown (left=treated tumor; right=untreated contralateral).

FIGS. 13A-13F. Tumor growth regression in mice treated with NS-TLR7a. BALB/c mice were implanted with CT26 cells and treatment began when tumors reached 100 mm³. Mice (n=5/group) were randomized and treated with vehicle (PBS), silica NS, unconjugated agonist (TLR7a), or NS-TLR7a every other day. Tumor tissues were collected on day 10 post treatment for analysis. A) Protocol of treatment. B) Average tumor growth curves per treatment. (C and D) Immunohistochemistry (IHC) images of CD3⁺ cells infiltrating into the tumor environments for (C) unconjugated TLR7a or (D) TLR7a-NS conjugates locally injected tumor. (E and F) IFN-γ gene expression level (E) and (F) quantified CD3⁺ T cells (F) in tumor samples for vehicle, NS, TLR7a, and TLR7a-NS conjugate treated groups (n=4-5/group). Representative IHC images were shown. One-way ANOVA was used for statistical significance analysis indicated as *=p<0.05, **=p<0.01, ***=p<0.001.

FIGS. 14A-14E. Plasma Cytokine Levels with 1V209 and 1V209-nanoshell conjugate treatments. A single dose of TLR7a (50 nmol/mouse), NS-TLR7a (50 nmol TLR7a; 1.8 mg NS), NS (1.8 mg/mouse) or vehicle was administered intratumorally. Blood was collected at 0, 2, and 24 hours and plasma was isolated. Cytokine levels for of IL-6, IL-12, IP-10, and MCP-1 were measured using a Luminex beads assay. One-way ANOVA compared to vehicle group was used for statistical significance analysis indicated as * (p<0.05), ** (p<0.01), *** (p<0.001), and **** (p<0.0001).

FIGS. 15A-15B. Biodistribution of NS-TLR7a. Indium¹¹¹ labeled NS-TLR7a were i.t. and i.v. injected into CT26 tumor bearing mice. Mice were bearing two tumors per mouse to observe the particle distribution. A) Scintigraphy images of NS-TLR7a i.t. and i.v. injected mice. Pure Indium¹¹¹ was placed in a tube at the scintigraphic plane as a positive control (on the left side of each scintigraphy image, as labeled). B) Mice were sacrificed and each organ was harvested for gamma counting at 72 hours after NS-TLR7a injection. Data were analyzed with 2 way ANOVA using Bonferroni post hoc test. n.s. indicates not significant and **** denotes p<0.0001.

FIGS. 16A-16H. Tumor progression curves and survival rates. CT26 colon tumor cells (1×10⁶) were implanted subcutaneously into female BALB/c mice (4-5 mice/group) at right and left flanks. Three therapies were performed to investigate the efficacy of combinational therapy and the treatment protocol is shown in A). Vehicle, simply combined checkpoint inhibitors (aPD1+aCTLA4), and checkpoint inhibitors combined with NS-TLR7a (aPD1+aCTLA4+NS-TLR7a) were chosen for longer-term tumor growth monitor. Both B)-D) treated tumors and E)-G) untreated tumors were monitored and measured. Checkpoint inhibitor (aPD-1 and aCTLA4) were injected i.p. and NS-TLR7a was injected i.t. 100 μg aPD1/aCTLA4 i.p three times per week and 12.5 nmol NS-TLR7a i.t. every other day. Mice were sacrificed when tumor reached 2,000 mm³ or ulceration. H) Survival study was investigated until day 90 after treatment and Logrank test was used for significance (** denotes as p<0.01).

FIGS. 17A-17B. Investigation of intratumorally injected NS-TLR7a Hematological and biochemistry effects on NS-TLR7a. Female BALB/c mice (n=4-5/group) were bearing two tumors on right and left flanks. NS-TLR7a or PBS were intratumorally injected every other day for total of 6 doses. Whole blood and sera were collected on day 14 post-treatment for complete blood count and biochemistry analysis. The investigation includes: A) red blood cells, white blood cells, and platelet. B) immune cells under white blood cell class. Data were analyzed by t-test with Welch's correction. n.s. indicates not significant and * denotes p<0.05.

FIGS. 18A-18I. Flow cytometry analysis of tumor microenvironment. NS-TLR7a, aPD1, and aCTLA4 were combined to treat two CT26 tumors-bearing mice and immune cell population in tumor microenvironment was investigated. A) Protocol of treatment. NS-TLR7a was i.t. injected into one tumor (treated tumor) and checkpoint inhibitors were i.p. injected. In treated tumor, the populations of B) CD45+, C) CD8+, D) IFN-γ+, and E) granzyme B+ cells were studied. Same investigation was done on untreated contralateral tumors F)-I). Immune cells population in treated and untreated tumors were studied. In each study group, n=5-6/group and has repeated twice. Data are shown one repeat study. Data were analyzed by Kruskal-Walls test using Dunn's multiple comparisons post hoc test. * means p<0.05 and ** means p<0.01.

FIGS. 19A-19C. Hematological and biochemistrical effects on intratumorally injected NS-TLR7a. Female BALB/c mice (n=4-5 mice/group) were intratumorally injected 6 doses of NS-TLR7a into one tumor on the flank, and the contralateral tumor was not injected. Whole blood and sera were collected on day 14 after first treatment. A) protein component, B) electrolytes, and C) hepatic and renal function indicator were shown. Data were analyzed by t-test with Welch's correction. n.s. indicates not significant and * denotes p<0.05.

FIGS. 20A-20B. Characterization of unmodified silica nanoshells and TLR7a (NS-TLR). Transmission electron microscopy images of (A) unmodified 100 nm silica shells and (B) 1V209 conjugated 100 nm silica nanoshells with high coating density. No morphological differences were observed.

FIGS. 21A-21B. UV-Vis characterization of NS-TLR. 100 nm nanoshells were conjugated with 1V209 at varying concentrations, re-suspended at 20 mg/mL (silica mass) and characterized using UV-Vis. (A) The amount of 1V209 conjugated for each formulation (low, medium, high density) was quantified by 1V209 absorbance peak at 283 nm. (bB) 1V209 UV-Vis standards used for NS-TLR quantification. 1V209 standards were prepared at different concentrations ranging from 14 μM-870 μM, and UV-Vis measurements were used to create a standard curve. The amount of 1V209 conjugated to the surface of the nanoshells using different formulations was quantified by using a standard curve.

FIGS. 22A-22C. Ligand quantification of NS-TLR via UV-vis background subtraction. 100 nm nanoshells were conjugated with 1V209 at varying concentrations, re-suspended at 20 mg/mL (silica mass) and characterized using UV-Vis. The amount of 1V209 conjugated for each formulation was quantified by 1V209 absorbance peak at 283 nm. (A) The absorbance spectrum of conjugates (high density coating) were fitted with a Rayleigh scattering curve. (B) A background subtraction was applied after quantification due to absorption from nanoparticles scattering. (C) Absorption spectra of NS-TLR after scattering background subtraction.

FIG. 23. In vitro cytokine induction of NS-TLR in murine cells. Mouse BMDCs (10⁵ cells) were plated and incubated with serially diluted 100 nm NS-TLR at different coating densities for 18 hours. IL-12 released in the culture supernatants of mouse BMDCs incubated with unconjugated 1V209, high, medium, low coating density NS-TLR, and silica shells. IL-12 cytokine was measured via ELISA. All data are representative dose response curves in mean±SD with triplicate technical repeats. Data are representative of three independent experiments showing similar results. Data were analyzed by two-way ANOVA with Tukey's post hoc analysis.

FIG. 24. In vitro IL-1β release of NS-TLR and mixture in BMDCs. 75-2500 nM of NS-TLR (high coating density) and mixture of silica nanoshells and 1V209 were incubated with wild type BMDCs for 18 hours. IL-1p cytokine was measured via ELISA. Dose response curves are in means SD. Data were analyzed by t test.

FIG. 25. Immunization study of NS-TLR with OVA as an antigen. IFN-γ secretion of culture supernatants of splenocytes incubated with NS-TLR (covalently bonded), mixture of 1V209 ligands with silica nanoshells (non-covalently bonded), unconjugated 1V209, silica nanoshells or vehicle. Splenocytes were incubated for 5 days at 37° C. with OVA protein and IFN-γ in supernatant was tested by ELISA.

FIG. 26. Synthesis and conjugation process of 100 nm silica nanoshells and TLR7 agonists, 1V209 Synthesis of 100 nm silica nanoshells. Silica nanoshells with —OH termination were first reacted with APTES to graft amine-groups onto the silica surface. Simultaneously, 1V209 was reacted with NHS and EDC to form an activated 1V209. EDC coupled NHS to carboxyls on 1V209, forming an NHS ester that is a stable intermediate. The activated NHS ester of 1V209 reacted with the primary amines on the silica surface of the shells. Note that APTES may have multiple linkage isomers to the surface and also among themselves or another APTES on binding to the silica surface. This figure shows a single surface Si—O—Si surface linkage isomer to simply represent bonding between APTES and silica nanoshells.

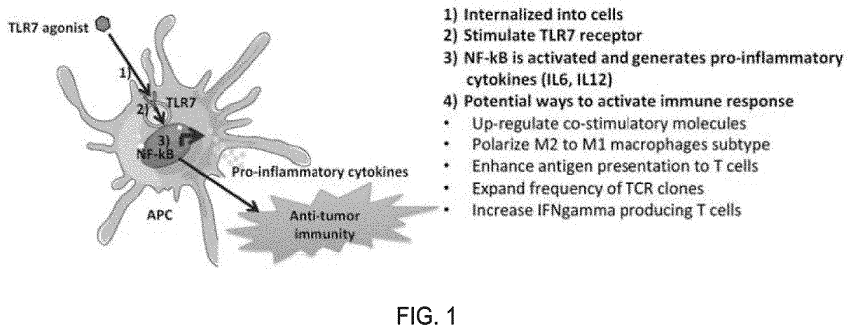

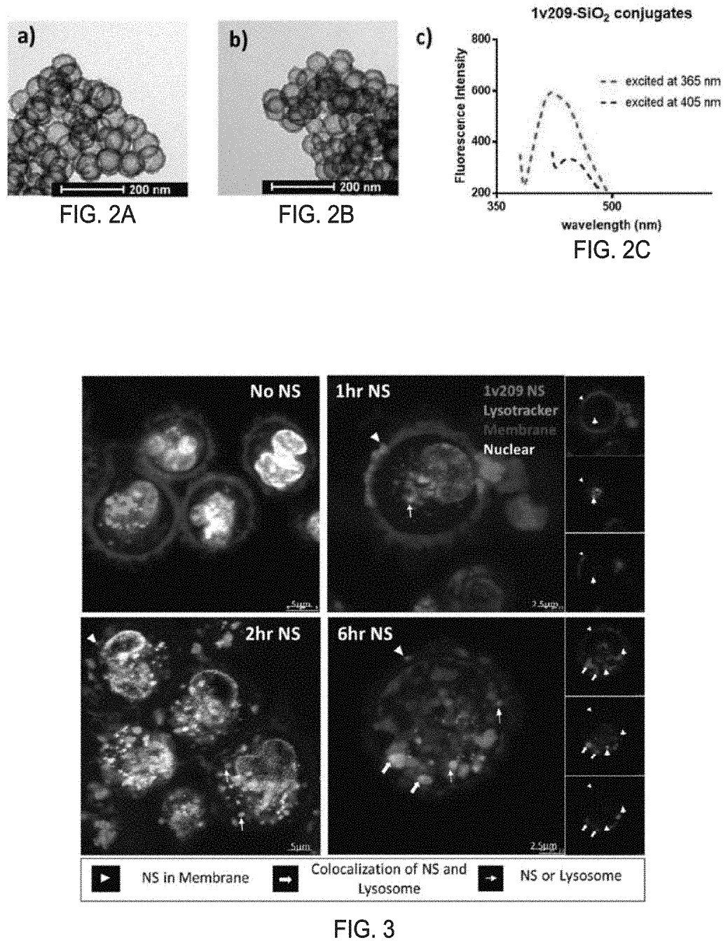

### DETAILED DESCRIPTION

Checkpoint inhibitors have brought potential to treat cancer, but only a minor population of patients have benefited from the single therapy checkpoint inhibitors due to insufficient immune response induction (Cogdill et al., 2017). Being one of top three cause of cancer death worldwide, colorectal cancer (CRC) is currently being investigated for immunotherapy (Cassidy & Syed, 2017). Metastatic CRC (mCRC) patients usually receive systemic therapy, such as anti-VEGF or anti-EGFR. However, such drugs often result in acquired resistance (Ciombro et al., 2015; Fakih, 2015). Checkpoint inhibitors, such as ipilimumab and nivolumab, have changed therapy possibilities for many tumors including melanoma, advanced lung and head and neck cancers, and are being evaluated to treat mCRC patients (Hodi et al., 2016). However, a large portion of patients did not respond to immune checkpoint inhibitors (Arora & Mahalingam, 2018; Emambux et al., 2018).

Based on the data from current clinical trials, patients whose tumors are mismatch repair (MMR) deficient are likely to respond toward checkpoint inhibitor therapies (Le et al., 2017; Overman et al., 2017; Le et al., 2015). It is potentially due to the DNA mismatch repair deficiency that would produce more neoantigen release that becomes easily recognized by the body's immune cells as neoantigen which in turn causes tumor-specific immune response. Pembrolizumab has recently been designated by the US Food and Drug Administration (FDA) for usage in MMR deficiency tumors, regardless of the tumor location (Boyiadzis et al., 2018). Even though immune checkpoint inhibitors have shown great promise for many cancer patients, MMR proficient cancer patients are less responsive (Le et al., 2015; Le 2016). Similarly, checkpoint inhibitors have shown great treatment effects in MMR deficiency mCRC that more than half of patients with stable disease control and at the same time the treatment demonstrated acceptable safety profile. Unfortunately, checkpoint inhibitors are not effective in MMR proficient cancer which has lower lymphocytes infiltration in tumors (Le et al., 2017), and so rendering these tumors sensitive to immunotherapy remains a major challenge (Emambux et al., 2018). Therefore, using immunostimulatory agents to stimulate immune response appears to be a promising approach to improve the cancer treatment with checkpoint inhibitor therapy. Murine colon cancer cell line, CT26, shares molecular features with aggressive human colorectal carcinoma cells, and is one of the most extensively used syngeneic mouse tumor models that lack mutation in mismatch repair genes (Castle et al., 2014; Efremova et al., 2018). CT26 colon tumor cell line was chosen for this study for the combination therapy of checkpoint inhibitors treatment improvement.

As described herein, TLR7 agonists (TLR7a) conjugated to 100 nm silica nanoshells (NS) with a high coating ligand density, termed NS-TLR7a, showed improved TLR7 activation. It is reflected by increased downstream pro-inflammatory cytokine IL-12 secretion, and an activated inflammasome pathway. Furthermore, NS-TLR7a enhanced and induced a Th1 biased immune responses that often associated with induction of cytotoxic T lymphocytes. Such T lymphocytes play a critical role in cancer immunotherapy because they can directly recognize and kill cancerous cells (Tosolini et al., 2011). In this study, NS-TLR7a was used to amplify body's immune response together with the checkpoint inhibitor to restore the T lymphocytes' killing capability. Such particle-based TLR therapy has the potential to treat MMR proficient colon cancer patients by increasing epitope recognition and development of tumor reactive T-cells at the tumor site.

To prolong the local effect of the TLR7 agonists, the small molecule TLR7 agonists have been conjugated to large molecules (Kim et al., 2016; Friedman et al., 2013). Herein, it is demonstrated that conjugation with silica particles enhances the agonistic activities and induces the inflammasome activation in addition to the innate immune activation by TLR7 signaling pathway.

TLR7 agonist and silica combination triggered NLRP3 inflammasome and released IL-1β to re-stimulate NF-kB pathway while neither unconjugated TLR7 agonist nor silica shells produced IL-1p. An immunization study demonstrated that TLR7 agonist-silica shells conjugates increased OVA-specific IgG antibodies a thousand-fold in mice sera and skewed the response to a Th-mediated immunity compared to unconjugated TLR7 agonists. When TLR7 agonist-silica shell conjugates were administered intratumorally into mice, the silica shells have a tendency of prolonging the agonist retention time. The localization of the agonist can avoid undesirable side effects and continue stimulating the NF-kB pathway, leading to the increase of the infiltration of T cells in tumors. The conjugates demonstrated an increased T cell infiltration and T lymphocyte-related gene upregulation compared to unconjugated TLR7 agonists or silica shells.

Exemplary TLR 7 agonists useful in the particles and methods of the invention include but are not limited to resiquimod (R-848), imiquimod, and synthetic TLR7 agonists, e.g., those of formula (II), e.g., 1V209 (2-methoxyethoxy-8-oxo-9-(4-carboxy benzyl)adenine), or SM360320 (9-benzyl-8-hydroxy-2-(2-methoxyethoxy) adenine).

Accordingly, there is provided a silica shell having a conjugated TLR7 agonist that is a compound of formula (I):

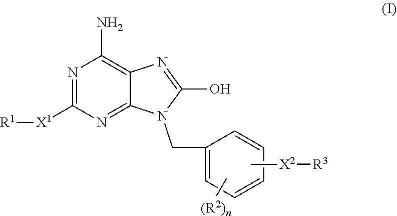

wherein X¹ is —O—, —S—, or —NR—;

wherein R¹ is hydrogen, (C₁-C₁₀)alkyl, substituted (C₁-C₁₀)alkyl, C₆₋₁₀aryl, substituted C₆₋₁₀aryl, C₅₋₉heterocyclic, or substituted C₅₋₉heterocyclic;

wherein R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

wherein each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent:

wherein each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

wherein n is 0, 1, 2, 3 or 4;

wherein X² is absent, (C₁-C₁₀)alkyl, substituted (C₁-C₁₀)alkyl, a bond or a linking group;

wherein R³ comprises a silica shell;

or a tautomer thereof;

or a pharmaceutically acceptable salt or solvate thereof.

In one embodiment, a silica shell or other nanoparticle is modified to comprise a compound of formula (II):

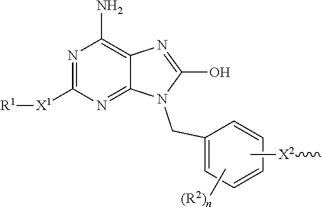

wherein X¹ is —O—, —S—, or —NR—;

wherein R₁ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

wherein R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

wherein each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

wherein each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;

wherein n is 0, 1, 2, 3 or 4;

wherein X² is absent, (C₁-C₁₀)alkyl, substituted (C₁-C₁₀)alkyl, a bond or a linking group;

or a tautomer thereof;

or a pharmaceutically acceptable salt or solvate thereof.

In one embodiment, the silica shell, e.g., having a diameter of about 100 nm, has less than about 1000 molecules of compound (II), about 1000 to about 6000 molecules of compound (II), or greater than about 6000 molecules, e.g., up to 10,000 to 30,000 molecules, e.g., about 10,000 to about 12,000, molecules, of compound (II).

In one embodiment, X¹ is —O— or —S—.

In one embodiment, R¹ is hydrogen (C₁-C₁₀)alkyl, substituted (C₁-C₁₀)alkyl, C₆₋₁₀aryl, or substituted C₆₋₁₀aryl.

In one embodiment, each R² is independently —OH, (C₁-C₆)alkyl, substituted (C₁-C₆)alkyl, (C₁-C₆)alkoxy, substituted (C₁-C₆)alkoxy, halo, nitro, or cyano, or R² is absent.

In one embodiment, X is —O—.

In one embodiment, wherein R¹ is (C₁-C₆)alkyl or substituted (C₁-C₆)alkyl.

In one embodiment, each R² is independently —OH or halo, or R² is absent.

In one embodiment, the silica shell has less than about 0.001, 0.05 or 0.03 TLR7 agonist molecules per nm², about 0.03 to 0.2 TLR7 agonist molecules per nm² or greater than about 0.2, e.g., up to 0.4 to 0.8 TLR7 agonist molecules per nm or 0.5 to 10 TLR7 agonist molecules per nm².

### Definitions

As used herein, the term “antibody” refers to a protein having one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad of immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V) and variable heavy chain (Vx) refer to these light and heavy chains respectively.

Antibodies may exist as intact immunoglobulins, or as modifications in a variety of forms including, for example, FabFc₂, Fab, Fv, Fd, (Fab′)₂, an Fv fragment containing only the light and heavy chain variable regions, a Fab or (Fab)□₂ fragment containing the variable regions and parts of the constant regions, a single-chain antibody, e.g., scFv, CDR-grafted antibodies and the like. The heavy and light chain of a Fv may be derived from the same antibody or different antibodies thereby producing a chimeric Fv region. The antibody may be of animal (especially mouse or rat) or human origin or may be chimeric or humanized. As used herein the term “antibody” includes these various forms.

A composition is comprised of “substantially all” of a particular compound, or a particular form a compound (e.g., an isomer) when a composition comprises at least about 90%, 95%, 99%, or 99.9%, of the particular composition on a weight basis. A composition comprises a “mixture” of compounds, or forms of the same compound, when each compound (e.g., isomer) represents at least about 10% of the composition on a weight basis. A purine analog of the invention, or a conjugate thereof, can be prepared as an acid salt or as a base salt, as well as in free acid or free base forms. In solution, certain of the compounds of the invention may exist as zwitterions, wherein counter ions are provided by the solvent molecules themselves, or from other ions dissolved or suspended in the solvent.

As used herein, the term “isolated” refers to in vitro preparation, isolation and/or purification of a nucleic acid molecule, a peptide or protein, or other molecule so that it is not associated with in vivo substances or is present in a form that is different than is found in nature. Thus, the term “isolated” when used in relation to a nucleic acid, as in “isolated oligonucleotide” or “isolated polynucleotide” refers to a nucleic acid sequence that is identified and separated from at least one contaminant with which it is ordinarily associated in its source. An isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. Hence, with respect to an “isolated nucleic acid molecule”, which includes a polynucleotide of genomic, cDNA, or synthetic origin or some combination thereof, the “isolated nucleic acid molecule” (1) is not associated with all or a portion of a polynucleotide in which the “isolated nucleic acid molecule” is found in nature, (2) is operably linked to a polynucleotide which it is not linked to in nature, or (3) does not occur in nature as part of a larger sequence. The isolated nucleic acid molecule may be present in single-stranded or double-stranded form. When a nucleic acid molecule is to be utilized to express a protein, the nucleic acid contains at a minimum, the sense or coding strand (i.e., the nucleic acid may be single-stranded), but may contain both the sense and anti-sense strands (i.e., the nucleic acid may be double-stranded).

The term “amino acid” as used herein, comprises the residues of the natural amino acids (e.g., Ala, Arg, Asn, Asp, Cys, Glu, Gin, Gly, His, Hyl, Hyp, lie, Leu, Lys, Met, Phe, Pro, Ser, Thr, Trp, Tyr, and Val) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, -methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an -methylbenzyl amide). Other suitable amino and carboxy protecting groups are known to those skilled in the art (see for example, T. W. Greene, Protecting Groups In Organic Synthesis; Wiley: New York, 1981, and references cited therein). For instance, an amino acid can be linked to the remainder of a compound of formula I through the carboxy terminus, the amino terminus, or through any other convenient point of attachment, such as, for example, through the sulfur of cysteine.

The term “toll-like receptor” (TLR) refers to a member of a family of receptors that bind to pathogen associated molecular patterns (PAMPs) and facilitate an immune response in a mammal. Ten mammalian TLRs are known, e.g., TLR1-10.

The term “toll-like receptor agonist” (TLR agonist) refers to a molecule that binds to a TLR. Synthetic TLR agonists are chemical compounds that are designed to bind to a TLR and activate the receptor. Exemplary synthetic TLR agonists provided herein include “TLR-7 agonist”, “TLR” agonist”, “TLR-3 agonist” and “TLR-9 agonist.” TLR agonists include imiquimod, resiquimod, broprimine and loxoribine.

The term “nucleic acid” as used herein, refers to DNA, RNA, single-stranded, double-stranded, or more highly aggregated hybridization motifs, and any chemical modifications thereof. Modifications include, but are not limited to, those providing chemical groups that incorporate additional charge, polarizability, hydrogen bonding, electrostatic interaction, and fluxionality to the nucleic acid ligand bases or to the nucleic acid ligand as a whole. Such modifications include, but are not limited to, peptide nucleic acids (PNAs), phosphodiester group modifications (e.g., phosphorothioates, methylphosphonates), 2′-position sugar modifications, 5-position pyrimidine modifications, 7-position purine modifications, 8-position purine modifications, 9-position purine modifications, modifications at exocyclic amines, substitution of 4-thiouridine, substitution of 5-bromo or 5-iodo-uracil; backbone modifications, methylations, unusual base-pairing combinations such as the isobases, isocytidine and isoguanidine and the like. Nucleic acids can also include non-natural bases, such as, for example, nitroindole. Modifications can also include 3′ and 5′ modifications such as capping with a BHQ, a fluorophore or another moiety.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media for example ether, ethyl acetate, ethanol, isopropanol, or acetonitrile. Lists of suitable salts are found in Remington's Pharmaceutical Sciences, 17th ed., Mack Publishing Company, Easton, Pa., p. 1418 (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Het can be heteroaryl, which encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine agonist activity using the standard tests described herein, or using other similar tests which are well known in the art. It is also understood by those of skill in the art that the compounds described herein include their various tautomers, which can exist in various states of equilibrium with each other.

“Therapeutically effective amount” is intended to include an amount of a composition useful in the present invention or an amount of the combination of compounds, e.g., to treat or prevent the disease or disorder, or to treat the symptoms of the disease or disorder, in a host. As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

As used herein, the term “patient” refers to organisms to be treated by the methods of the present invention. Such organisms include, but are not limited to, mammals such as humans. In the context of the invention, the term “subject” generally refers to an individual who will receive or who has received treatment (e.g., administration of a composition of the invention).

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

In cases where compounds are sufficiently basic or acidic to form acid or base salts, use of the compounds as salts may be appropriate. Examples of acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Alkyl includes straight or branched C₁₋₁₀ alkyl groups, e.g., methyl, ethyl, propyl, butyl, pentyl, isopropyl, isobutyl, 1-methylpropyl, 3-methylbutyl, hexyl, and the like.

Lower alkyl includes straight or branched C₁₋₆ alkyl groups, e.g., methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, and the like.

The term “alkylene” refers to a divalent straight or branched hydrocarbon chain (e.g., ethylene: —CH₂—CH₂—).

C₃₋₇ Cycloalkyl includes groups such as, cyclopropyl, cyclopentyl, cyclohexyl, cycloheptyl, and the like, and alkyl-substituted C₃₋₇ cycloalkyl group, e.g., straight or branched C₁₋₆ alkyl group such as methyl, ethyl, propyl, butyl or pentyl, and C₅₋₇ cycloalkyl group such as, cyclopentyl or cyclohexyl, and the like.

Lower alkoxy includes C₁₋₆ alkoxy groups, such as methoxy, ethoxy or propoxy, and the like.

Lower alkanoyl includes C₁₋₆ alkanoyl groups, such as formyl, acetyl, propanoyl, butanoyl, pentanoyl or hexanoyl, and the like.

C₇₋₁₁ aroyl, includes groups such as benzoyl or naphthoyl:

Lower alkoxycarbonyl includes C₂₋₇ alkoxycarbonyl groups, such as methoxycarbonyl, ethoxycarbonyl or propoxycarbonyl, and the like.

Lower alkylamino group means amino group substituted by C₁₋₆ alkyl group, such as, methylamino, ethylamino, propylamino, butylamino, and the like.

Di(lower alkyl)amino group means amino group substituted by the same or different and C₁₋₆ alkyl group (e.g., dimethylamino, diethylamino, ethylmethylamino).

Lower alkylcarbamoyl group means carbamoyl group substituted by C₁₋₆ alkyl group (e.g., methylcarbamoyl, ethylcarbamoyl, propylcarbamoyl, butylcarbamoyl).

Di(lower alkyl)carbamoyl group means carbamoyl group substituted by the same or different and C₁₋₆ alkyl group (e.g., dimethylcarbamoyl, diethylcarbamoyl, ethylmethylcarbamoyl).

Halogen atom means halogen atom such as fluorine atom, chlorine atom, bromine atom or iodine atom.

Aryl refers to a C₆₋₁₀ monocyclic or fused cyclic aryl group, such as phenyl, indenyl, or naphthyl, and the like.

Heterocyclic or heterocycle refers to monocyclic saturated heterocyclic groups, or unsaturated monocyclic or fused heterocyclic group containing at least one heteroatom, e.g., 0-3 nitrogen atoms (—NR^{d}— where R^{d} is H, alkyl, or Y² as defined herein), 0-1 oxygen atom (—O—), and 0-1 sulfur atom (—S—). Non-limiting examples of saturated monocyclic heterocyclic group includes 5 or 6 membered saturated heterocyclic group, such as tetrahydrofuranyl, pyrrolidinyl, morpholinyl, piperidyl, piperazinyl or pyrazolidinyl. Non-limiting examples of unsaturated monocyclic heterocyclic group includes 5 or 6 membered unsaturated heterocyclic group, such as furyl, pyrrolyl, pyrazolyl, imidazolyl, thiazolyl, thienyl, pyridyl or pyrimidinyl. Non-limiting examples of unsaturated fused heterocyclic groups includes unsaturated bicyclic heterocyclic group, such as indolyl, isoindolyl, quinolyl, benzothizolyl, chromanyl, benzofuranyl, and the like. A Het group can be a saturated heterocyclic group or an unsaturated heterocyclic group, such as a heteroaryl group.

R^{c} and R¹ taken together with the nitrogen atom to which they are attached can form a heterocyclic ring. Non-limiting examples of heterocyclic rings include 5 or 6 membered saturated heterocyclic rings, such as 1-pyrrolidinyl, 4-morpholinyl, 1-piperidyl, 1-piperazinyl or 1-pyrazolidinyl, 5 or 6 membered unsaturated heterocyclic rings such as 1-imidazolyl, and the like.

The alkyl, aryl, heterocyclic groups of R¹ can be optionally substituted with one or more substituents, wherein the substituents are the same or different, and include lower alkyl; cycloalkyl, hydroxyl; hydroxy C₁₋₆ alkylene, such as hydroxymethyl, 2-hydroxyethyl or 3-hydroxypropyl; lower alkoxy; C₁₋₆ alkoxy C₁₋₆ alkyl, such as 2-methoxyethyl, 2-ethoxyethyl or 3-methoxypropyl; amino; alkylamino; dialkyl amino; cyano; nitro; acyl; carboxyl; lower alkoxycarbonyl; halogen; mercapto; C₁₋₆ alkylthio, such as, methylthio, ethylthio, propylthio or butylthio; substituted C₁₋₆ alkylthio, such as methoxyethylthio, methylthioethylthio, hydroxyethylthio or chloroethylthio; aryl; substituted C₆₋₁₀ monocyclic or fused-cyclic aryl, such as 4-hydroxyphenyl, 4-methoxyphenyl, 4-fluorophenyl, 4-chlorophenyl or 3,4-dichlorophenyl; 5-6 membered unsaturated heterocyclic, such as furyl, pyrrolyl, pyrazolyl, imidazolyl, thiazolyl, thienyl, pyridyl or pyrimidinyl; and bicyclic unsaturated heterocyclic, such as indolyl, isoindolyl, quinolyl, benzothiazolyl, chromanyl, benzofuranyl or phthalimino. In certain embodiments, one or more of the above groups can be expressly excluded as a substituent of various other groups of the formulas.

In some embodiments, the five-membered ring of the formula is a thiazole ring.

The alkyl, aryl, heterocyclic groups of R² can be optionally substituted with one or more substituents, wherein the substituents are the same or different, and include hydroxyl; C₁₋₆ alkoxy, such as methoxy, ethoxy or propoxy; carboxyl; C₂₋₇ alkoxycarbonyl, such as methoxycarbonyl, ethoxycarbonyl or propoxycarbonyl) and halogen.

The alkyl, aryl, heterocyclic groups of R^{c} can be optionally substituted with one or more substituents, wherein the substituents are the same or different, and include C₃₋₆ cycloalkyl; hydroxyl; C₁₋₆ alkoxy; amino; cyano; aryl; substituted aryl, such as 4-hydroxyphenyl, 4-methoxyphenyl, 4-chlorophenyl or 3,4-dichlorophenyl; nitro and halogen.

The heterocyclic ring formed together with R^{c} and R¹ and the nitrogen atom to which they are attached can be optionally substituted with one or more substituents, wherein the substituents are the same or different, and include C₁₋₆ alkyl; hydroxy C₁₋₆ alkylene; C₁₋₆ alkoxy C₁₋₆ alkylene; hydroxyl; C₁₋₆ alkoxy; and cyano.

A specific value for R¹ is hydrogen, C₁₋₄alkyl, or substituted C₁₋₄alkyl.

Another specific R¹ is 2-hydroxyethyl, 3-hydroxypropyl, 4-hydroxybutyl, 2-aminoethyl, 3-aminopropyl, 4-aminobutyl, methoxymethyl, 2-methoxyethyl, 3-methoxypropyl, ethoxymethyl, 2-ethoxyethyl, methylthiomethyl, 2-methylthioethyl, 3-methylthiopropyl, 2-fluoroethyl, 3-fluoropropyl, 2,2,2-trifluoroethyl, cyanomethyl, 2-cyanoethyl, 3-cyanopropyl, methoxycarbonylmethyl, 2-methoxycarbonylethyl, 3-methoxycarbonylpropyl, benzyl, phenethyl, 4-pyridylmethyl, cyclohexylmethyl, 2-thienylmethyl, 4-methoxyphenylmethyl, 4-hydroxyphenylmethyl, 4-fluorophenylmethyl, or 4-chlorophenylmethyl.

Another specific R is hydrogen, CH₃—, CH₃—CH₂—, CH₃CH₂CH₂—, hydroxyC₁₋₄alkylene, or C₁₋₄alkoxyC₁₋₄alkylene.

Another specific value for R¹ is hydrogen, CH₃—, CH₃—CH₂—, CH₃—O—CH₂CH₂— or CH₃—CH₂—O—CH₂CH₂—.

A specific value for R² is hydrogen, halogen, or C₁₋₄alkyl.

Another specific value for R² is hydrogen, chloro, bromo, CH₃—, or CH₃—CH₂—.

Specific substituents for substitution on the alkyl, aryl or heterocyclic groups are hydroxy, C₁₋₆alkyl, hydroxyC₁₋₆alkylene, C₁₋₆alkoxy, C₁₋₆alkoxyC₁₋₆alkylene, C₃₋₆cycloalkyl, amino, cyano, halogen, or aryl.

A specific value for X² is a chain having up to about 12 atoms; wherein the atoms are selected from the group consisting of carbon, sulfur, and non-peroxide oxygen.

A specific value for X² is a carboxyl group.

A specific value for X² is a bond or a chain having up to about 24 atoms; wherein the atoms are selected from the group consisting of carbon, nitrogen, sulfur, non-peroxide oxygen, and phosphorous.

Another specific value for X² is a bond or a chain having from about 4 to about 12 atoms.

Another specific value for X² is a bond or a chain having from about 6 to about 9 atoms.

Another specific value for X² is

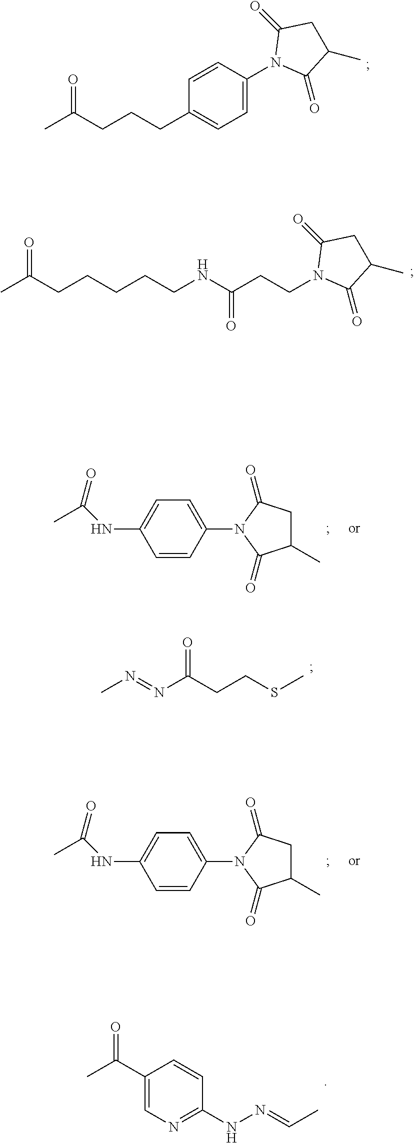

Another specific value for X² is

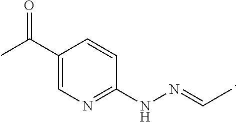

The compositions of this invention are administered in a therapeutically effective amount to a subject in need of treatment. Administration of the compositions of the invention can be via any of suitable route of administration, particularly parenterally, for example, intravenously, intra-arterially, intraperitoneally, intrathecally, intraventricularly, intraurethrally, intrasternally, intracranially, intratumorally, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the compositions may be formulated as a sterile solution in water or another suitable solvent or mixture of solvents. The solution may contain other substances such as salts, sugars (particularly glucose or mannitol), to make the solution isotonic with blood, buffering agents such as acetic, critric, and/or phosphoric acids and their sodium salts, and preservatives.

The compositions of the invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it may include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, methods of preparation are vacuum drying and the freeze drying techniques, which yield a powder of the active ingredient plus any additional desired ingredient present in the previously sterile-filtered solutions.

For topical administration, the present compositions may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compositions can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the invention provides various dosage formulations of the conjugates for inhalation delivery. For example, formulations may be designed for aerosol use in devices such as metered-dose inhalers, dry powder inhalers and nebulizers.

Examples of useful dermatological compositions which can be used to deliver the compositions of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compositions of the invention can be determined by comparing their in vitro activity, and in vivo activity in animal models. Methods for the extrapolation of effective dosages in mice, and other animals, to humans are known to the art; for example, see U.S. Pat. No. 4,938,949. The ability of a composition of the invention to act as a TLR agonist may be determined using pharmacological models which are well known to the art, including the procedures disclosed by Lee et al., Proc. Natl. Acad. Sci. USA, 100: 6646 (2003).

Generally, the concentration of the composition(s) of the invention in a liquid composition, such as a lotion, will be from about 0.1-25 wt-%, or from about 0.5-10 wt-%. The concentration in a semi-solid or solid composition such as a gel or a powder will be about 0.1-5 wt-%, or about 0.5-2.5 wt-%.

The active ingredient may be administered to achieve peak plasma concentrations of the active compound of from about 0.5 to about 75 μM, about 1 to 50 μM, or about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, in the range of 6 to 90 mg/kg/day, or in the range of 15 to 60 mg/kg/day.

The composition is conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual patient. In general, the total daily dose range for a compound or compounds of formula (I), for the conditions described herein, may be from about 50 mg to about 5000 mg, in single or divided doses. A daily dose range may be about 100 mg to about 4000 mg, or about 1000-3000 mg, in single or divided doses, e.g., 750 mg every 6 hr of orally administered composition. This can achieve plasma levels of about 500-750 uM, which can be effective to kill cancer cells. In managing the patient, the therapy should be initiated at a lower dose and increased depending on the patient's global response.

As described above, compositions are useful in the treatment or prevention of a disease or disorder in, for example, humans or other mammals (e.g., bovine, canine, equine, feline, ovine, and porcine animals), and perhaps other animals as well. Depending on the particular compound, the composition will, for example, be useful for treating cancer, an infection, enhancing adaptive immunity (e.g., antibody production, T cell activation, etc.), as vaccines, an adjuvant, and/or stimulating the central nervous system.

### EXEMPLARY EMBODIMENTS

In one embodiment, a silica shell, the exterior of which is modified to allow for covalent linkage of one or more distinct TLR7 agonists. In one embodiment, the shell has a diameter less than about 200 nm. In one embodiment, the shell has a diameter less than about 150 nm. In one embodiment, the shell has a diameter less than about 100 nm. In one embodiment, the shell has a diameter greater than about 20 nm. In one embodiment, the shell has a diameter greater than about 50 nm. In one embodiment, the TLR7 agonist comprises formula (II). In one embodiment, X in formula (II) is —O—, —S—, or —NR^{c}—; R¹ in formula (II) is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic; R^{c} in formula (II) is hydrogen, C₁₋₁₀alkyl, or substituted C₁₋₁₀alkyl; or R^{c} and R¹ taken together with the nitrogen to which they are attached form a heterocyclic ring or a substituted heterocyclic ring; each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent; each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl; wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl; n is 0, 1, 2, 3 or 4; and X² in formula (II) is absent, (C₁-C₁₀)alkyl, substituted (C₁-C₁₀)alkyl, a bond or a linking group; or a tautomer thereof; or a pharmaceutically acceptable salt or solvate thereof. In one embodiment, the TLR7 agonist comprises 1V209 or SM360320. In one embodiment, the composition further comprises a checkpoint inhibitor, e.g., pembrolizumab, nivolumab, atezolizumab, avelumab, durvalumab, or ipilimumab. In one embodiment, R2 in formula (II) comprises (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, substituted aryl(C₁-C₆)alkyl, alkoxy substituted C6 aryl, Het, Het (C₁-C₆)alkyl, or (C₁-C₆)alkoxycarbonyl. In one embodiment, R2 in formula (II) comprises H, —C₁-C₆ alkyl, —C₁-C₆ alkoxy, —NR^{a}R^{b}, —N₃, —OH, —CN, —COOH, —COOR¹, —C₁-C₆ alkyl-NR^{a}R^{b}, C₁-C₆ alkyl-OH, C₁-C₆ alkyl-CN, C₁-C₆ alkyl-COOH, C₁-C₆ alkyl-COOR¹, 5-6 membered ring, substituted 5-6 membered ring, —C₁-C₆ alkyl-5-6 membered ring, —C₁-C₆ alkyl-substituted 5-6 membered ring C₂-C₉ heterocyclic, or substituted C₂-C₉ heterocyclic. In one embodiment, X in formula (II) comprises —C₁-C₆ alkyl, —C₁-C₆ alkoxy, —NR^{a}R^{b}, —N₃, —OH, —CN, —COOH, —COOR¹, —C₁-C₆ alkyl-NR^{a}R^{b}, C₁-C₆ alkyl-OH, C₁-C₆ alkyl-CN, C₁-C₆ alkyl-COOH, C₁-C₆ alkyl-COOR¹, 5-6 membered ring, substituted 5-6 membered ring, —C₁-C₆ alkyl-5-6 membered ring, —C₁-C₆ alkyl-substituted 5-6 membered ring C₂-C₉ heterocyclic, or substituted C₂-C₉ heterocyclic.

In one embodiment, a method to inhibit or treat cancer is provided. In one embodiment, the method includes administering to a mammal in need thereof an effective amount of the composition having silica shells conjugated to one or more TLR7 agonist, optionally in conjunction with one or more chemotherapeutics or anti-cancer antibodies. In one embodiment, the mammal is a human. In one embodiment, the method further includes administering a checkpoint inhibitor, e.g., pembrolizumab, nivolumab, atezolizumab, avelumab, durvalumab, or ipilimumab. In one embodiment, the composition is parenterally administered. In one embodiment, the composition is intravenously administered. In one embodiment, the composition is locally administered. In one embodiment, the composition is intratumorally administered. In one embodiment, the method further comprises employing ultrasound guided histotripsy. In one embodiment, the amount administered enhances pro-inflammatory cytokines. In one embodiment, the amount administered enhances pro-co-stimulatory molecule expression in dendritic cells. In one embodiment, the cancer is bladder cancer, prostate cancer, testicular cancer, lung cancer, breast cancer, brain cancer, liver cancer, ovarian cancer, kidney cancer, or pancreatic cancer.

In one embodiment, a composition having the silica shells conjugated to a TLR7 agonist may be employed as an anti-viral.

The invention will be described by the following non-limiting examples.

### Example 1

### Rationale and Results

Toll-like receptor (TLR) recognizes pathogen-associated molecular pattern (PAMPs) and induces host defense response. TLR ligands have obtained attention as being an adjuvant in cancer treatment. Signaling through TLRs can result in cytokine production and polarization of CD4⁺ helper and CD8⁺ effector T cells. It was hypothesized that conjugating small molecule TLR7 ligands onto nano- to micro-sized silica shells would facilitate the uptake of the agonist by mimicking how PAMPs on viral particles are recognized by antigen presenting cells (APCs). The synthetic TLR7 ligand, 1V209, contains a carboxyl benzyl moiety that can be modified easily and therefore was chosen. In order to have efficient intracellular delivery in phagocytic antigen presenting cells, small molecules are often conjugated to macromolecules to enhance endocytosis (Bachmann et al., 2010). Hollow silica nanoshells have low toxicity and high capability of surface modification, and are therefore being selected as a TLR ligands carrier (Yu et al., 2011). Preliminary data showed that the combination of ovalbumin (OVA) protein, TLR7 agonist/mouse serum albumin with silica can induce most strong immune response compared to polystyrene beads or protein alone.

1V209-nanoshell conjugates have fluorescent properties with an emission peak at 421 nm or 446 nm when excited with a wavelength of 325 nm or 405 nm, respectively. Although the fluorescence intensity is lower when excited with a 405 nm laser, this characteristic is useful because it allows for 1V209-nanoshell conjugates visualization with a fluorescence microscope equipped with a 405 nm laser.

TLR7 is localized in the endosomal compartment (Scho et al., 2008), therefore trafficking of 1V209-nanoshell conjugates into the endosomal compartment of dendritic cells (DCs) is important in order for the ligand to engage with the toll-like receptor and thus stimulate DCs. Thus, we tested 1V209-silica shell conjugation influencing 1V209 uptake by DCs. BMDCs were incubated with 1V209-nanoshell conjugates and imaged overtime. Due to 1V209's fluorescent properties, no further fluorescence labeling was required to track the 1V209-nanoshell conjugates. This property is a substantial advantage, considering that adding another fluorescent label can alter the cell interaction with the nanoshell conjugates. As shown in the FIG. 3, nanoshell conjugates began to interact with the cell membrane within initial 1 hour. After 2 hours, 1V209-nanoshell conjugates were trafficked into the cytosol compartment, and the majority of nanoshell conjugates were in the late endosome/lysosome compartments at 6-hour post-treatment. These results show that 1V209-nanoshell conjugates rapidly become associated with the cell membrane of BMDCs; subsequently the conjugates are transported into the cytoplasm at 2 hours post-treatment, and the majority are localized within the endosome/lysosome compartment 6 hours post-treatment.

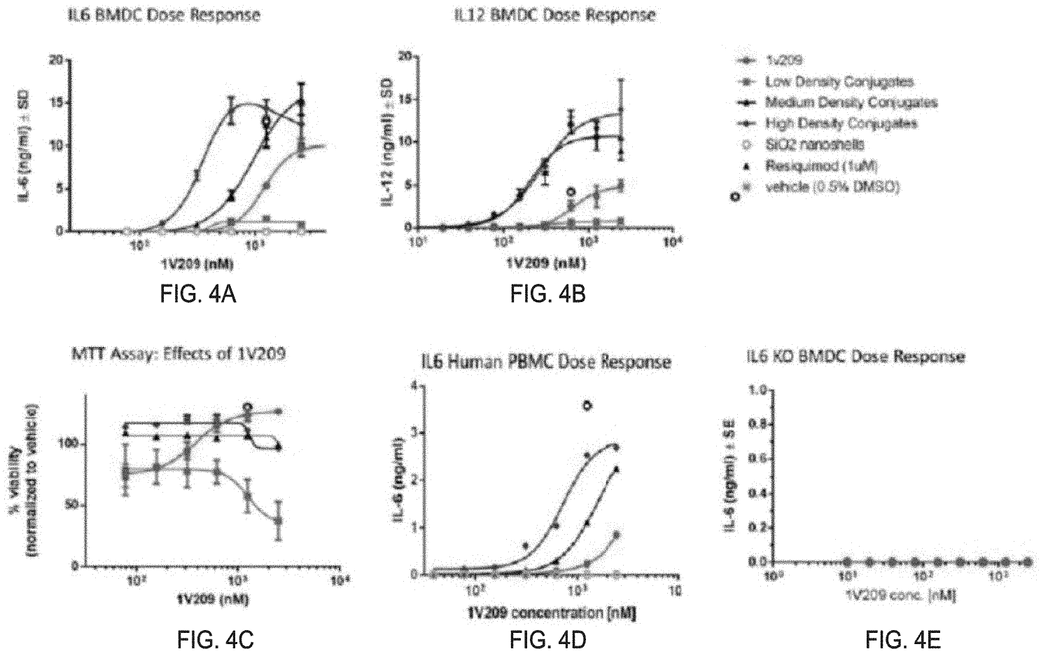

The correlation between TLR7 ligands density and cytokines induction level was further investigated. To test this, mouse bone marrow-derived dendritic cells (BMDCs) or human peripheral blood mononuclear cells (PBMCs) were treated with unconjugated 1V209, SiO₂ nanoshells, or 1V209-nanoshell conjugates with different coating densities (high, medium, or low coating densities). The levels of IL-6 and IL-12 release in the culture supernatant were measured. As shown in FIG. 3, when 1V209 was conjugated to silica nanoshells at a medium or high coating density, the agonistic activity of the TLR7 ligand was enhanced in both human PBMCs and mouse BMDCs (FIG. 4 A, B, D). High density and medium density coating conjugates appear to have higher potencies than unconjugated 1V209. The maximum efficacy (Emax) of 1V209-nanoshell conjugates at a high density coating is 2.9 times higher than unconjugated 1V209. Different formula of conjugates were also tested their cytotoxicity in BMDCs. Cells treated with SiO₂ nanoshells had reduced viability when unmodified or when conjugated with a low density, compared to medium and high coating densities. To determine whether the immune potency of 1V209-nanoshell conjugates was TLR7 specific, conjugates was performed in TLR7 deficient BMDCs. Cytokine induction assays showed no detectable levels of IL-6 from TLR7 knockout BMDCs (FIG. 4E). These results indicate that high density coating 1V209-nanoshell conjugates have the highest efficacy and low toxicity compared to unconjugated 1V209 and to the other formulations. Thus, high coating of TLR7 agonists silica shells are denoted as 1v209-NS conjugates in the following description.

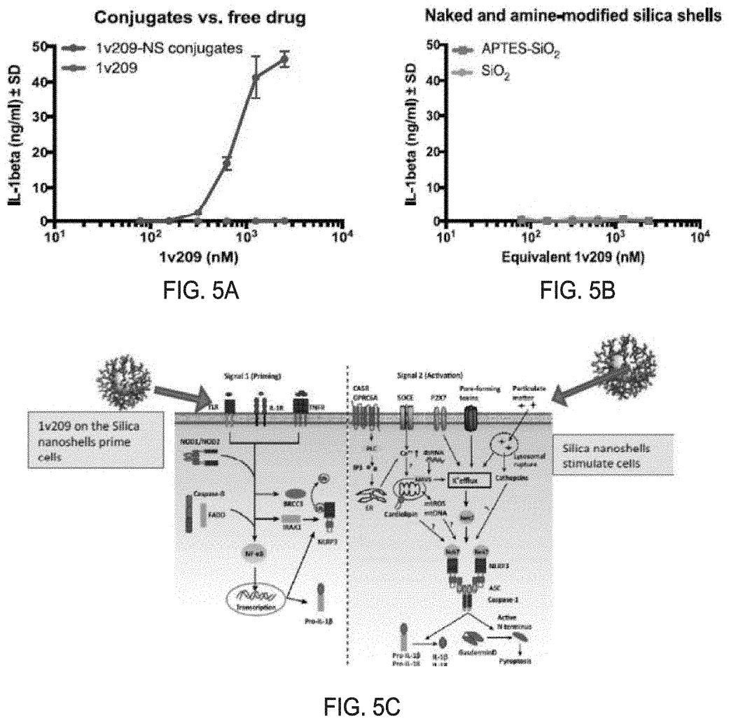

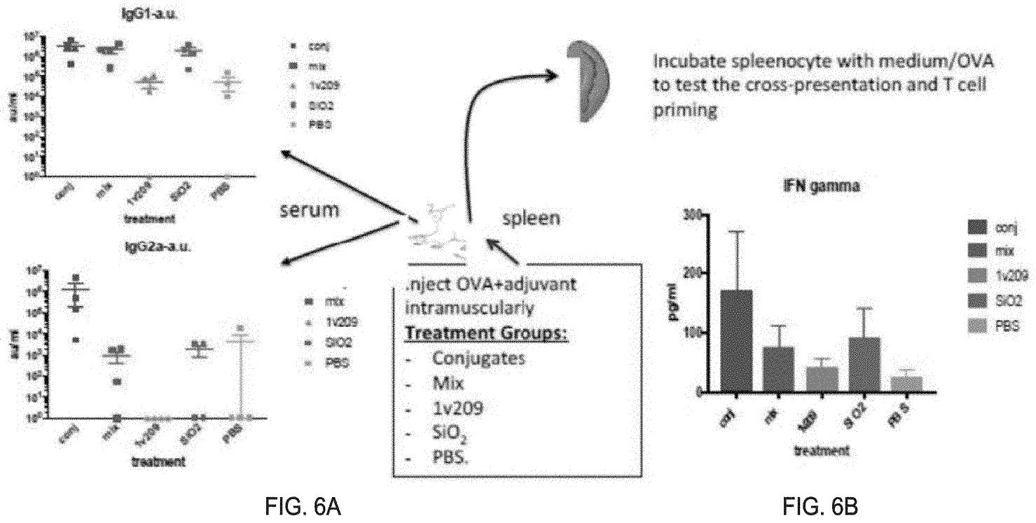

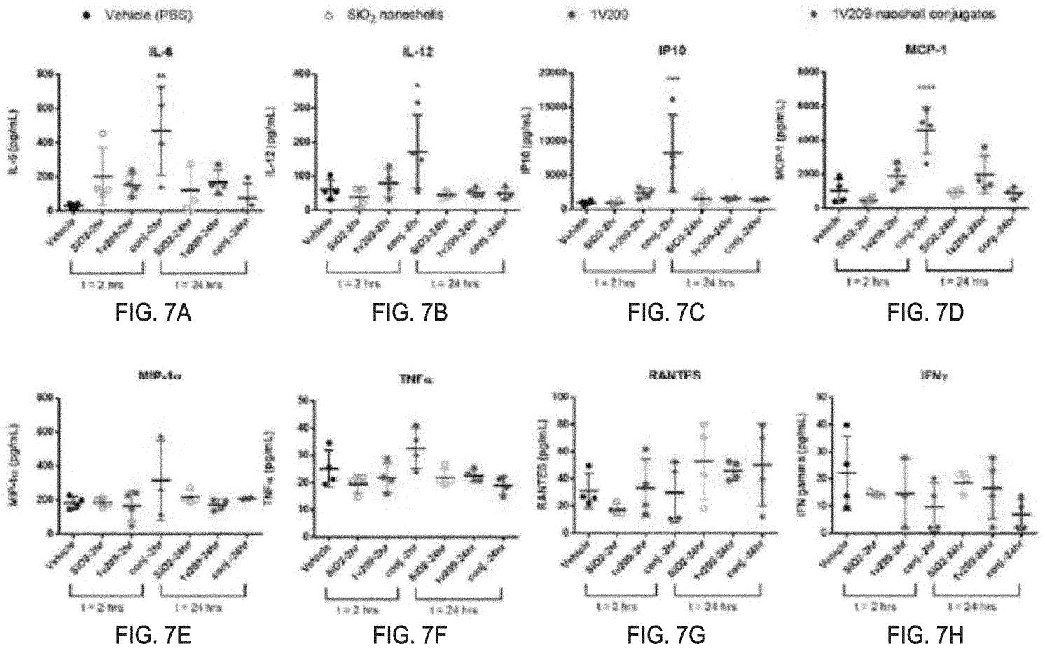

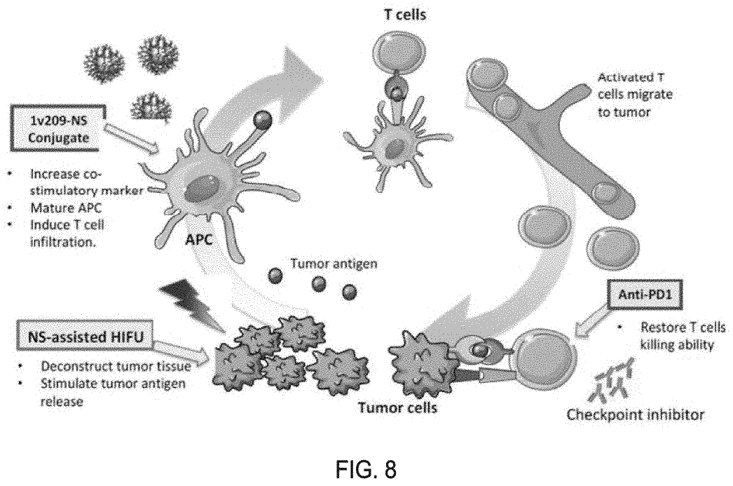

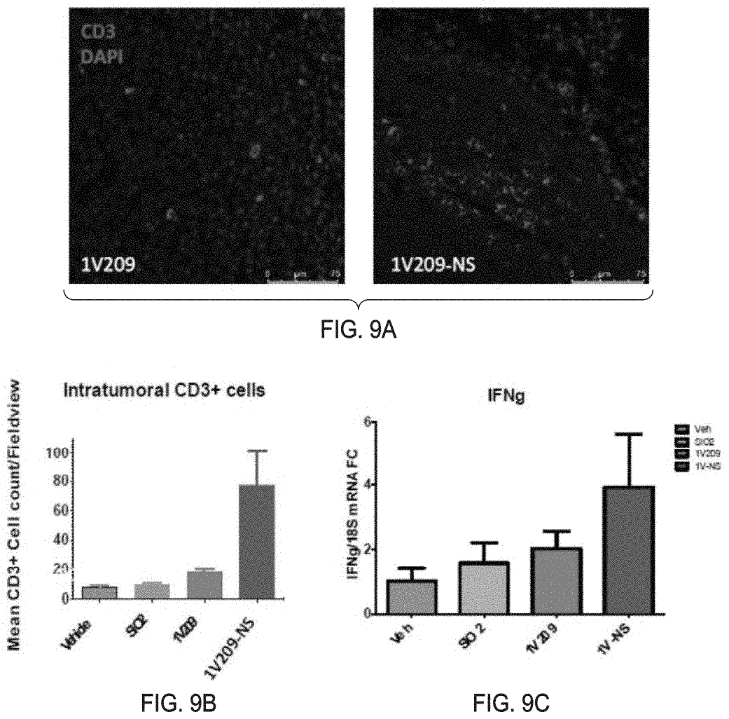

1V209-silica shells conjugates generate high levels of IL-1p, while free 1V209, naked silica shells, and amine modified silica shells (APTES-SiO₂ in the FIG. 5A-C) induce very low levels or zero IL-1β. IL-1β secretion by dendritic cells is mandatory for T cell priming (Ghiringhelli et al., 2009). A two-pronged pathway has been proposed for NLRP3 inflammasome activation in macrophages and DCs. In this model, the first signal is triggered by microbial or endogenous molecules that generate NLRP3 and pro-IL-1p expression through activation of NF-κB; the second signal is triggered by ATP, pore-forming toxins, viral RNA and particulate matters such as silica nanoparticles. It is likely that 1V209 triggers NF-kB pathway and produces pro-1β (inactive form) and silica shells stimulate cells to form NLRP3 inflammasome and cleave pro-IL-1β into IL-1β (active form) and release. Resting DCs/macrophages have insufficient NLRP3 in the cells, and therefore, incubating with NLRP3 activator (silica nanoshells) results in no or minimal IL-1β.

The stimulation of a T helper cell (Th) response plays a critical role in cancer immunotherapy (Zanetti, 2015; Knutson et al., 2005). Th1 response is associated with the induction of IgG2a and Th2 response is associated with IgG1. Th1 cells release IFN-γ which activates APC which contribute to increased antigen presentation to cytotoxic T lymphocytes (CTL). 1V209-silica shells conjugates show an increase OVA-specific antibodies 1,000-fold higher compared to unmodified silica shells. 1V209-silica shells also increase the frequency of IFN-γ producing T cells in spleen than unconjugated 1V209.

To evaluate innate immune activation by intratumoral (i.t.) administration of 1V209-silica shells conjugates, a systemic cytokine release study was performed to determine the cytokine levels in the blood after local administration. A single dose of 1V209 (50 nmol/mouse), silica shell conjugates with equivalent 1V209 dose, SiO₂ or PBS was i.t. administered and blood was collected at 0, 2, and 24 hours. Macrophage 1 (M1) polarization promotes Th1-skewed response which can lead to antitumor responses. M1 responses are characterized by secretion of cytokines such as IL-12, IL-6, TNFα, IL-1, IL-26, and RANTES, among others (Ka et al., 2014). Th1 cells are characterized by the secretion of cytokines such as IFN-γ, TNF-α, monocyte chemotactic protein-1 (MCP-1), and macrophage inflammatory protein-1α (MIP1α) (Kim et al., 2014). As shown in FIG. 5A-C, there was a statistically significant increase of IL-6, IL-12, IP10, and MCP-1 plasma samples collected from 1V209-silica shells conjugates after 2 hours. After 24 hours, all treated samples returned to basal levels. These data demonstrate that 1V209-silica shells conjugates induced a potent but transient innate immune response, which returns to basal levels within 24 hours.

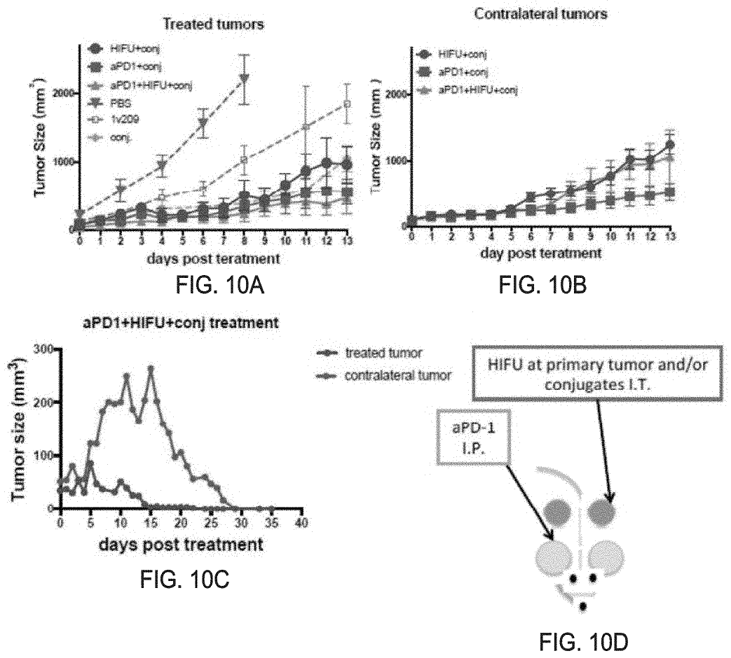

As shown in FIG. 10A-D, there was a higher degree (10-fold) of infiltrating lymphocytes (CD3⁺ cells) in tumors harvested from the mice treated with 1V209-silica shell conjugates (1V-NS in FIG. 10A-D) compared to the other treatment groups. These data demonstrated that conjugating TLR7 agonists to silica shells promotes T cell recruitment to the tumor, which has the potential to improve anti-tumor responses.

1V209-silica conjugates have been combined with non-thermal high intensity focused ultrasound (HIFU and non-thermal HIFU=histotripsy) and anti-PD1 (aPD1 in FIG. 10A-D). HIFU was applied at 1.1 MHz, 2% duty cycle and a 15-20 MPa focal pressure to ablate one side of tumor. 2 μm perfluoropentane (PFP) liquid filled silica microshells were intratumorally injected to enhance and focus the HIFU ablation specifically at the tumor site. To assess the systemic immune response, each mouse bears two tumors and only one site of tumor is treated. Combination therapy shows effective tumor growth inhibition on both treated and untreated tumors. HIFU may allow enhancement of therapy effects. One mouse from aPD1+HIFU+conj. treatment group shows full remission.

Combination therapy (TLR7 agonists-silica shells conjugates+anti-PD1+HIFU) treatment increased the lymphocyte related transcripts in both directly treated tumors and indirectly treated contralateral tumors.

By combing nanoshells-enhanced TLR7 agonist (conj. in FIG. 10A-D) with immune checkpoint inhibitor (a-PD1), it can slow down the tumor progression for both directly treated tumor and indirectly treated distant tumor. The addition of HIFU can further induce tumor remission for both treated site and untreated site of tumors. These results demonstrated that the TLR7 agonists-silica shells can enhance and induce the systemic anti-tumor immune response.

These TLR7 agonists-silica shells conjugates may be further modified as follows: to enhance cell internalization and particle localization, the TLR7 agonist may be conjugated to different sizes of particles for more selective tumor cells uptake and better localization, and/or particles may be modified with molecules, e.g., mannose, to be better internalized by antigen presenting cells.

In addition to, or as an alternative to, anti-PD1 therapy, e.g., with anti-PD1 antibodies, or HIFU, TLR7 agonist-silica shell conjugates may be combined with agents including but not limited to other checkpoint inhibitors (e.g., anti-CTLA 4, or anti-PD-L1), e.g., to de-activate Treg and restore CD4⁺, CD8⁺ T cells activities; oxaliplatin or other agents to induce immunogenic tumor cell death and activate TLR4; oncolytic virotherapy (virus transfection to kill tumor cells and release tumor antigens, the triggered immune response can be further amplified by TLR7 agonist conjugates); and/or epigenetic agents such as methyltransferases inhibitor (e.g., rrx-001, 5-AZA) that can transform M2 to M1 subtypes and release pro-inflammatory. It is hypothesized the methyltransferase inhibitors can activate macrophages through TLR4 activation. It is possible that the synergy effects can be achieved by combining epigenetic agents and 1V209-silica shell conjugates. Together stimulating TLR4 and TLR7 can largely amply the production of IL-12 and recruit more cytotoxic T cells to attack tumor cells.

Thus, the compositions disclosed herein are useful as adjuvants for vaccines or therapeutic agents for cancer treatment. By prolonging the accumulation time of TLR7 agonists locally via silica shells, the TLR7 agonist can enhance the therapeutic index of the unconjugated TLR7 agonists. The IL-1β induced by the TLR7 ligands-silica shells may be able to initiate the immune response and further amplify it. Thus, the TLR7 agonist-silica shell conjugates may be used to target the non- or low immunogenic tumor types. The combination therapy of TLR7 agonist-silica shells, checkpoint inhibitor and focused ultrasound also showed that it can induce a systemic tumor antigen specific immune response. This shows the potential of capability of targeting the metastatic tumor models. Additionally, the hollow core of the silica shells may be filled with perfluoropentane gas to be used as imaging contract agents or as ablative tools, giving them multifunctional properties.

### Example 2

### Methods and Materials

Materials: Diethylenetriamine (DETA, Cat. No. D93856), Tetramethyl orthosilicate (TMOS), trimethoxy(phenyl)silane (TMPS), N-Hydroxysuccinimide (NHS, Cat. No. 130672), N-(3-Dimethylaminopropyl)-N′-ethylcarbodiimide (EDC), and solvents were purchased from Sigma Aldrich (St. Louis, Mo.). 100 nm polystyrene templates were purchased from Polysciences Inc. (Warrington, Pa.). 2-(4-Isothiocyanatobenzyl)-diehtylenetriaminepentaaccetic acid (DTPA) was purchased from Macrocyclics (Dallas, Tex.). ¹¹¹InCl₃ was purchased from Covidien (Mansfield, Mass.). 4-[6-Amino-2-(2-methoxyethoxy)-8-oxo-7H-purin-9(8H)-yl]methylbenzoic acid (1V209) was synthesized as described in Chan et al. (2009). Anti-mouse PD-1 (CD279) antibodies (clone RMP1-14, Cat. No. BP0146) or anti-mouse CTLA-4 (CD152) antibodies (clone 9D9, Cat. No. BP0164) were purchased from BioXcell (West Lebanon, N.H.). RPMI Medium 1640 (Cat. No. 11875-093, Gibco) or DMEM (Cat. No. 15-013-CV, Corning) were supplemented with 10% fetal bovine serum (Cat. No. 35-011-CV, Corning) and 100 U/ml penicillin, 100 ug/ml Streptomycin, 292 μg/ml Glutamine (Cat. No. 10378-016, Life Technologies) to prepare complete media (RPMI-10 or DMEM-10).

Animals and Tumor Model: Mouse colon cancer cell line CT26 (Cat. No. CRL-2638) was purchased from American Type Culture Collection (ATCC). Six-8 week old female BALB/c mice were purchased from The Jackson Laboratory. 10⁶ cells/50 μL in PBS were injected into right and left flanks subcutaneously, and treatment was started at tumor size approximately 100 mm³. Tumor volume was determined by caliper with the formula: volume (mm³)=(width×width×length)/2. All procedures and protocols were approved by the UC San Diego Institutional Animal Care and Use Committee.

Histological Analysis: Tissue samples were fixed in 10% formalin (1 part of stock formaldehyde (37-40%) and 9 part of water) and transferred to 70% ethanol prior paraffin block processing and sectioning. Immunohistochemistry was done using rat anti-CD3 antibody (1:200, Cat. No. ab11089, Abcam). Images were obtained using a 10× dry objective on a SP8 Leica confocal microscope. Cell count analysis was performed using ImageJ and Leica proprietary software.

Preparation of tissue lysates: CT26 derived tumor tissues from in vivo study were collected and flash frozen to −80° C. for storage. Tissue lysates were prepared with a Next Advance NA-01 Tissue Homogenizer, in isolation buffer (Sucrose (MW 342.3) 70 mM; Mannitol (MW 182.2) 190 mM; HEPES pH 7-8 20 mM; EDTA pH 8 0.2 mM) supplemented with proteases and phosphatases inhibitors (1:100 dilution; Cat. No. 535140, P5726, Sigma-Aldrich) and RNASE-free homogenization beads (Cat. No. SSB14B and SSB32, Next-Advance).

RNA extraction and RT-qPCR expression analysis: Total RNA was extracted from cells and/or tissues lysates using the Quick-RNA Miniprep Kit (Cat. No. 11-328, Zymo Research), according to manufacturer's instructions, and reverse transcribed using the iScript cDNA Synthesis Kit (Cat. No. 170-8891, Bio-Rad). qPCR was done using SYBR Green and results were analyzed using the ΔΔCq method (Livak & Schmittgen, 2001) and normalized to housekeeping genes 18S and GAPDH. Primer sequences used were designed using NCBI's Primer BLAST and spanned exon-exon junctions. A list of used primer sequences is presented in Table 1.

TABLE 1

| Primer | | |
|---|---|---|
| Name | Forward (5′-3′) | Reverse (3′-5′) |
| IFN-γ | TTCTICCAGCAACAGCA | TCAGCAGCGACTCCTTTTCC |
| | AGGC(SEQ ID NO: 4) | (SEQ ID NO: 1) |
| 18S | CGAGCCGCCTGGATACC | CAIGGCCTCAGTICCGAAAA |
| | (SEQ ID NO: 5) | (SEQ ID NO: 2) |
| GAPDH | TCAAGCTCATTTCCTGG | TAGGGCCICTCTTGCTCAGT |
| | TATGACA(SEQ ID | (SEQ ID NO: 3) |
| | NO: 6) | |

Biodistribution: NS-TLR7a was prepared as previously described. 1 mL of 3 mg/mL of NS-TLR7a was functionalized with 2 μL of 1 mg/mL DTPA and pulse-vortexed for 24 hours. Functionalized NS-TLR7a was washed and re-suspended in 0.1M citrate buffer (pH=6) to 2 mg/mL solution. 2 mg of the nanoshells were incubated with about 100 μC of indium-111 chloride for 30 minutes. Radiolabeled NS-TLR7a was washed twice with buffer and twice with MilliQ purified water. During the wash procedure, the In111-labeled nanoshells and the supernatant were measured by dose calibrator to track the In111 retention. After washing, In111-NS-TLR7a was resuspended to 4 mg/mL in MilliQ purified water for injection in vivo. 100 μL of In111-NS-TLR7a was injected into two-tumors-bearing mice i.v. or i.t. Mice were imaged via planar scintigraphy immediately, 8, 24, 48, 72 hours post-injection. After 72 hours, mice were sacrificed and spleen, lung, heart, right tumor (injected), left tumor (uninjected), kidney, and liver were collected and counted gamma intensity.

Analysis of Tumor-Infiltrating Immune Cells: Each mouse had two CT26 tumors at right and left flanks. 12.5 nmol of NS-TLR7a in 50 μL was injected i.t. every other day for total of 6 doses (day 1, 3, 5, 7, 9, 11 after treatment). 200 μg of checkpoint inhibitor, anti-mouse PD-1 (aPD1) antibodies or anti-mouse CTLA-4 (aCTLA4) antibodies was injected i.p. three times weekly (day 0, 3, 6, 9, 12, 14 after treatment). Mice were sacrificed on day 14 for tumor infiltrating lymphocytes analysis. Tumors were dissociated into cell suspension using a mouse tumor dissociation kit with the gentleMACS Octo Dissociator according to the manufacturer's protocol (Miltenyi Biotec). Cell suspensions were incubated and stained with cocktails of anti-mouse CD45 (Cat. No. 103114, BioLengend) and anti-mouse CD8 (Cat. No. 48-0081, Invitrogen) antibodies at 4° C. for 30 minutes. Fixation/Permeabilization Solution kits were used for intracellular IFN-γ staining (Cat. No. 17-7311, BD Biosciences) and Granzyme B (Cat. No. 515403, BioLegend).

Statistical analysis: Statistical comparisons of continuous variables between groups was performed using two-way ANOVA followed by a Bonferroni or Dunnet post hoc test with GraphPad Prism software 5.0. To compare cross-sectional outcomes among more than two groups, Kruskal-Wallis tests with Dunn's post hoc test were applied. p<0.05 were considered statistically significant.

### Introduction

In this study, the efficacy of intratumoral administration of NS-TLR7a as a monotherapy in murine syngeneic colon cancer models was investigated and it was found that it increased the T cell infiltration in the colon tumor microenvironment and up-regulated the IFN-γ expression. Compared to unconjugated small molecule TLR7 agonists alone, NS-TLR7a has enhanced the cytokine induction in sera and resume to the safety baseline after 24 hours. The toxicology studies further indicated that the NS-TLR7a has a limited toxicity that no significant difference in complete blood cell count and hepatic function compared to vehicle. The NS-TLR7a has been proved that they are stationary after injection and can escape from spleen clearance. Although NS-TLR7a is retained at the local injection site, NS-TLR7a has the ability to induce a robust systemic tumor antigen-specific immune response. By using anti-PD1 and anti-CTLA4 together, NS-TLR7a can better inhibit tumor growth by increasing the number of infiltrating immune cells. The triple combination therapy including NS-TLR7a, anti-PD1 and anti-CTLA4 has the ability to induce both injected and contralateral tumors into full remission and has shown an improved survival rate. Therefore, NS-TLR7a has shown the potential to be an enhancer for current immunotherapy and improve the outcome of cancer treatment.

### Results and Discussion

### NS-TLR Exhibits Anti-Tumor Responses and Improved Survival

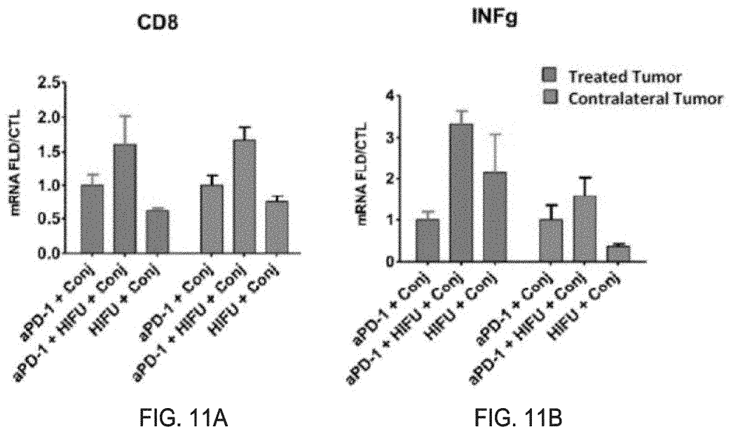

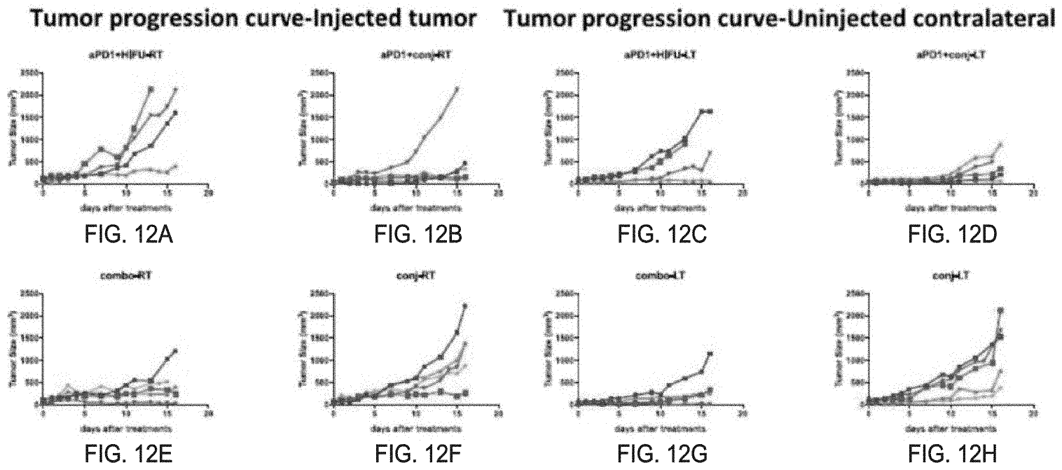

To test the anti-tumor effects of TLR7 with or without conjugation onto silica nanoshells in vivo, silica NS, unconjugated TLR7a (TLR7 agonist), and NS-TLR7a were tested in a CT26 mouse tumor model. For this study, mice were implanted with CT26 cells, a modestly immunogenic mouse colon cancer model, and treatment began when tumors reached 100 mm³. Tumor-bearing mice were treated with TLR7a (12.5 nmol), NS-TLR7a (12.5 nmol TLR7a; 0.44 mg silica nanoshells), silica NS (0.44 mg) or PBS intratumorally (i.t.) every other day up to 8 days (FIG. 11A). As shown in FIG. 11B, the group treated with NS-TLR7a exhibited a significant tumor growth regression compared to the vehicle or silica nanoshells treated group. On day 8, tumors were harvested for immunohistochemistry (IHC) analysis and CD3⁺ cells were assessed as T cell population in the tumor microenvironment.

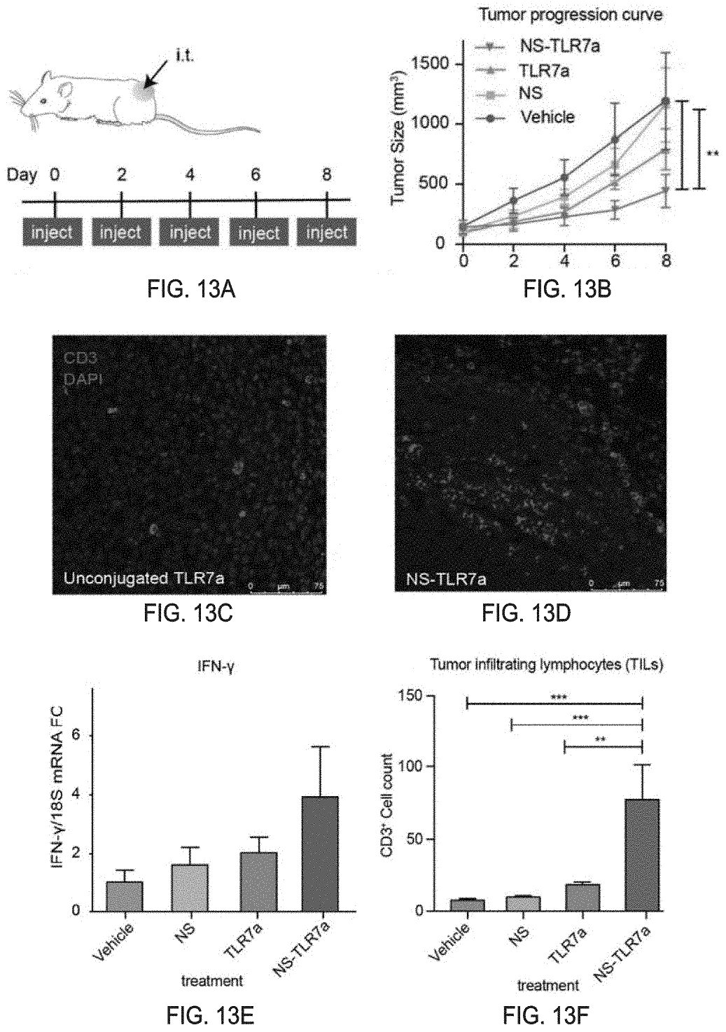

As shown in FIGS. 13C and 13D, TLR7 ligands conjugated onto silica nanoshells demonstrated a higher T cell infiltration compared to unconjugated TLR7 ligands, which is likely due to the quick clearance of small molecule TLR7 ligand drug that failed to be retained for long enough to active DC and subsequent adaptive immunity. FIGS. 13E and 13F demonstrated the quantified number of CD3⁺ T cells and IFN-γ gene expression in harvested tumor, showing TLR7 conjugated onto silica nanoshells exhibits a more robust immune response. The higher IFN-γ expression in NS-TLR7a treated group implies that a higher tumor killing activity occurring at tumor site when treated with NS-TLR7a compared to vehicle, silica nanoshells, or unconjugated TLR7 agonists. This data indicated that conjugating small molecule drugs onto silica nanoshells can improve the in vivo therapeutic effect and modulate the immune cell composition in the tumor. NS-TLR7a enhanced a transient systemic cytokine release response

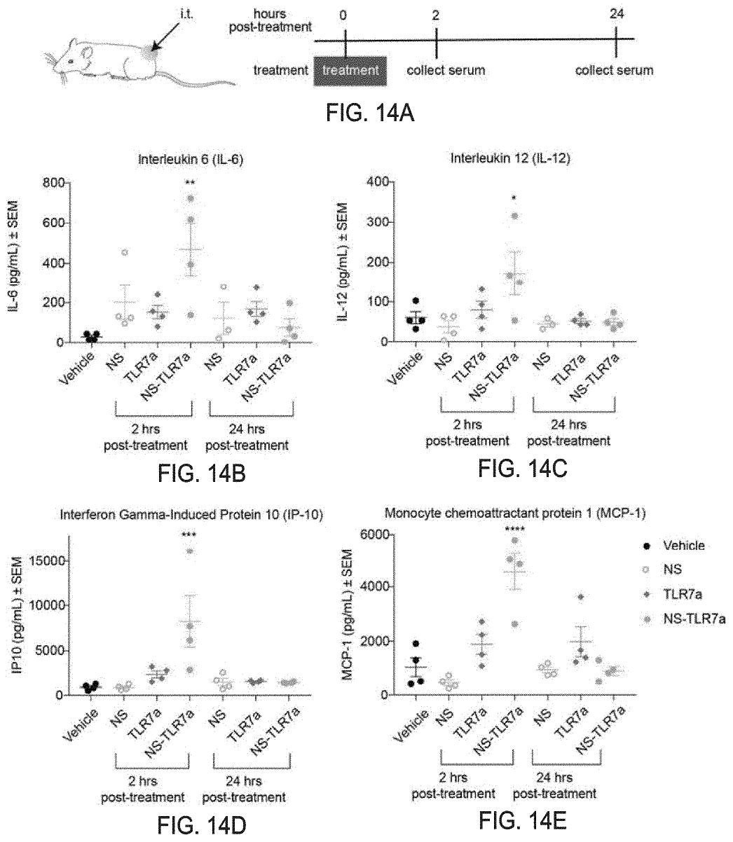

Additionally, a systemic cytokine release study was performed to determine the cytokine levels in the blood. A single dose of TLR7a (50 nmol/mouse), NS-TLR7a (50 nmol TLR7a; 1.76 mg NS), NS (1.76 mg/mouse) or vehicle was administered intratumorally and blood was collected at 0, 2, and 24 hours after injection. As shown in FIG. 14A-E, there was a statistically significant increase of IL-6, IL-12, IP-10, and MCP-1 plasma samples collected from NS-TLR7a 2 hours post-treatment. After 24 hours, all treated samples returned to basal levels. No weight loss adverse effects were observed for any of the treatment groups at the given dose. This data shows that NS-TLR7a can induce a potent transient innate immune response which returns to basal levels.

### I.T. Injected NS-TLR7a Remains at the Local Tumor Site and Avoid Nanoparticle Sequestration by Spleen

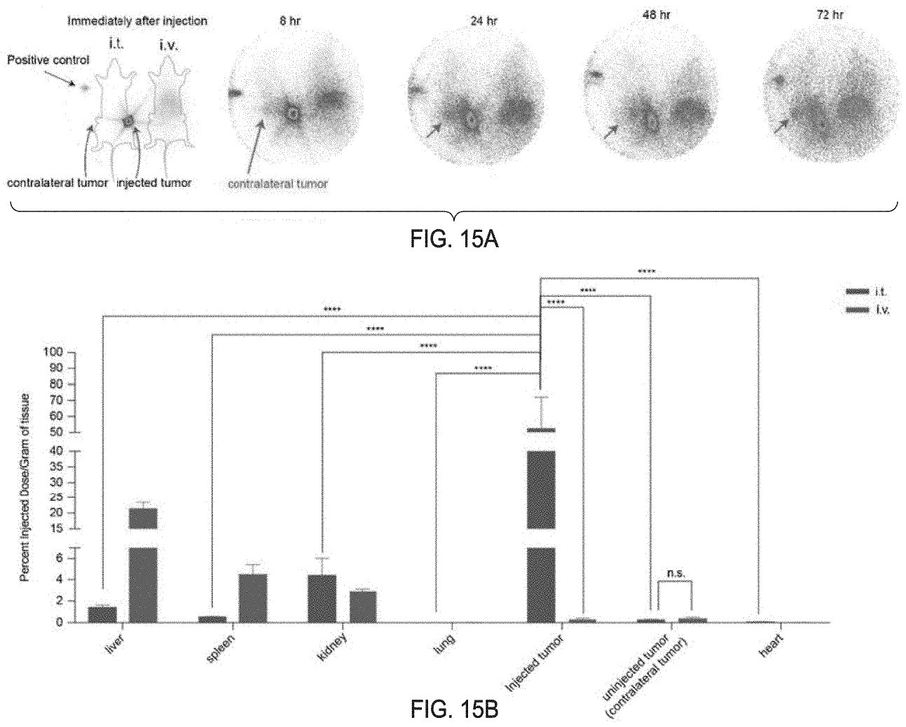

The biodistribution of the locally versus systemically administrated NS-TLR7a were investigated. NS-TLR7a was labeled with radioactive In¹¹¹ and injected i.t. or i.v. into mice. Each mouse bears two subcutaneous CT26 tumors at right and left flanks while only one tumor was injected with drugs. Planar γ-scintigraphy was used to monitor the particle distribution over 72 hours (FIG. 15A). Subsequently, organ biodistribution was performed by measuring organ radioactivity at 72-hour post-injection (FIG. 15B). Scintigraphy images were shown in FIG. 15A, as expected for most nanoparticle-delivery systems, systemically injected (i.v.) group showed nanoparticle accumulation in the reticuloendothelial system organs such as liver and spleen (Duan & Li, 2013; Brown et al., 2018; Yang et al., 2017)). Right after injection, these i.v. injected nanoparticles quickly start accumulating at the reticuloendothelial system and such phenomena continued over the time course. On the contrary, i.t. injected nanoparticles retained at the injected tumor site over 72 hours and only a small amount of nanoparticles accumulated at spleen, liver, or kidney. A small amount of i.t. injected nanoparticles traveled to the contralateral tumor, as early as 24 hours after i.t. injection (FIG. 15A, red arrow pointing the contralateral uninjected tumor). At 72-hour, organs were harvested for gamma counting to quantify the nanoparticles in each organ. As shown in FIG. 15B, systemically injected group showed that nanoparticles mainly accumulated at liver (21.4%) and spleen (4.5%). A small portion of nanoparticles (0.33%) traveled to contralateral tumor site that is likely due to the enhanced permeability and retention effect (EPR effect). At the same time, locally injected nanoparticles mainly retained at the injected tumor (52.4%), and there was a low amount of nanoparticles that accumulated at liver (1.4%) or spleen (0.55%). A similarly small amount of locally injected nanoparticles has traveled to the distant tumor (0.29%) to that systemically injected control, suggesting that EPR effects were not altered by the initial administration routes. The low incident nanoparticle tumoral accumulation indicates that EPR effect from nanoparticles has a minimal contribution to the drug potency improvement. However, lengthened retention time with the locally concentrated drug may improve the outcome of the agents. This result implies nanoshells can prolong the retention time of TLR7 agonists locally at the injection site. This may further amplify the immune response induced by TLR7 activation because the drug can continuously stimulate and drive dendritic cells to maturation. The lower amount of nanoparticle uptake at liver and spleen suggested that locally sustained NS-TLR7a can activate the immune innate system that enhances the tumor antigen presentation, resulting in triggering a strong adaptive immune response.

### Toxicology Analysis of NS-TLR7a

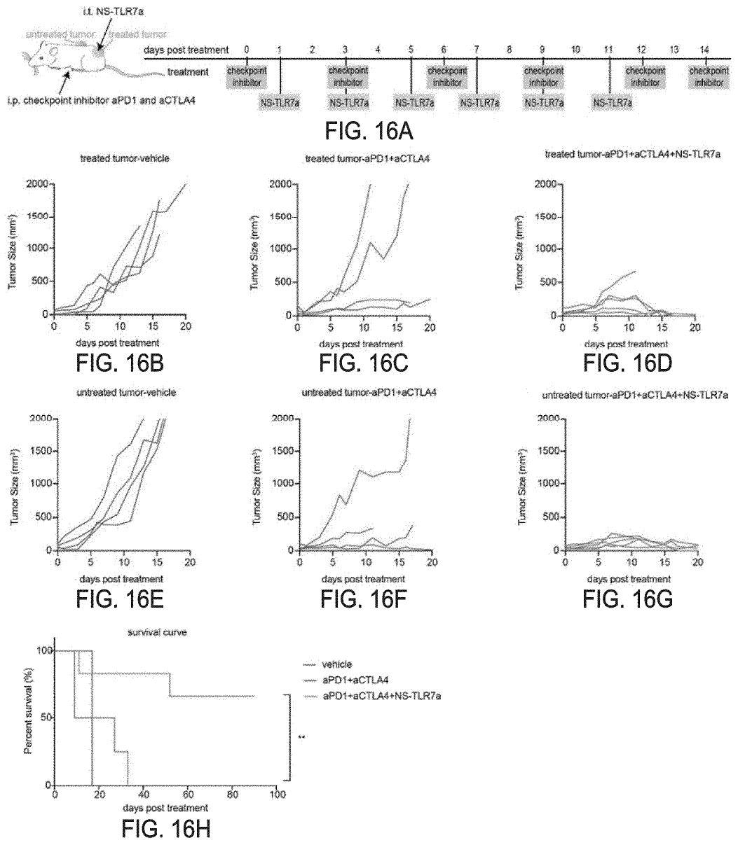

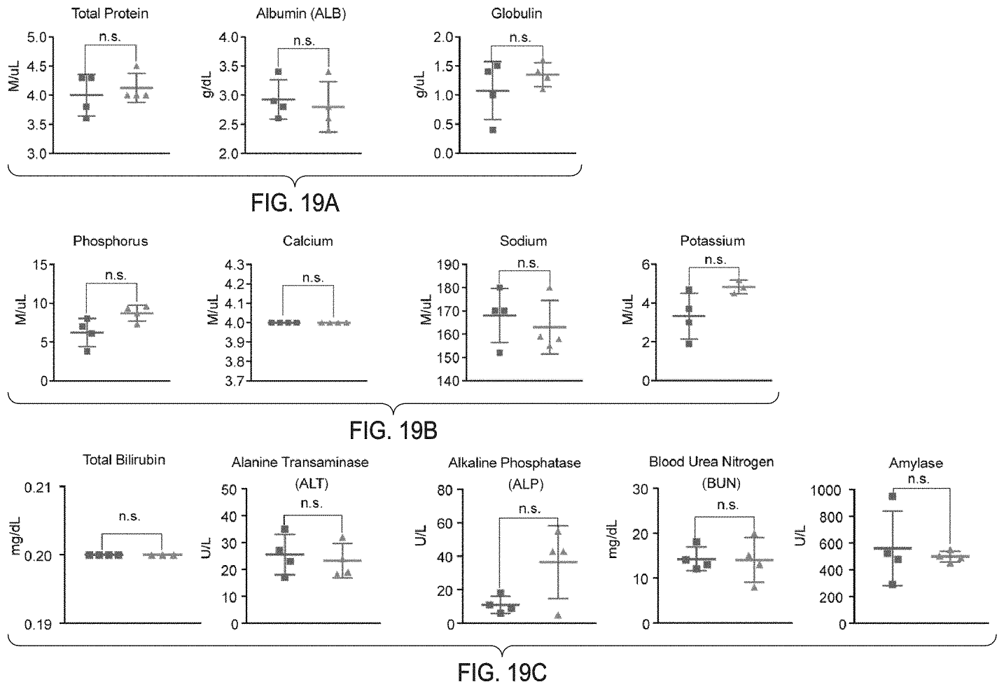

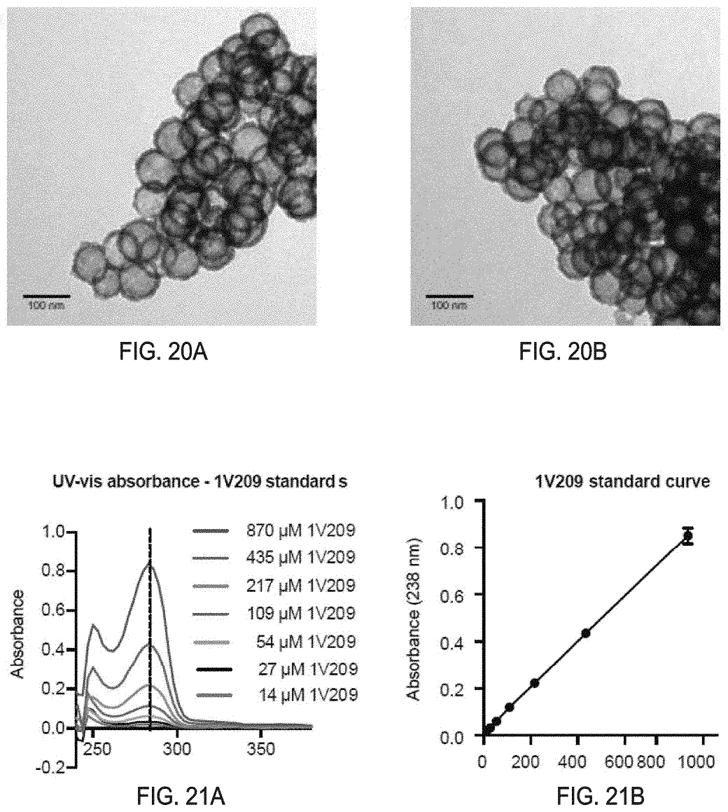

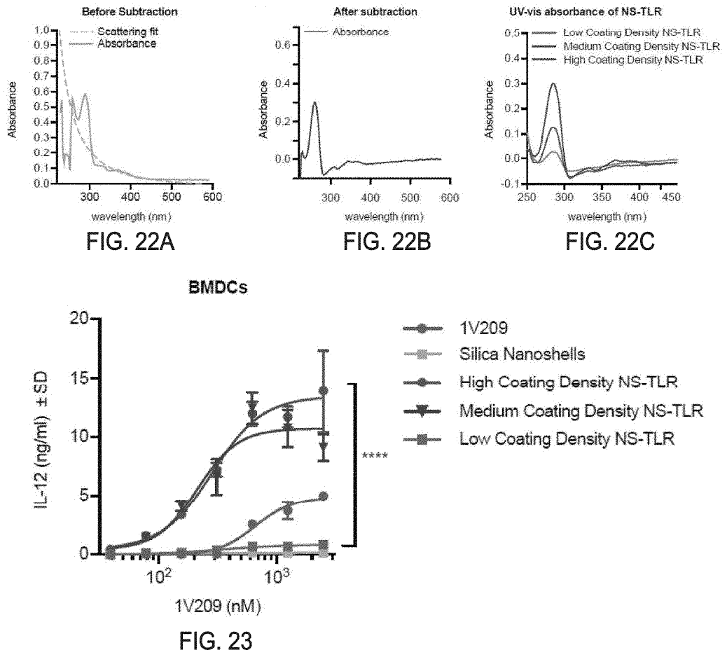

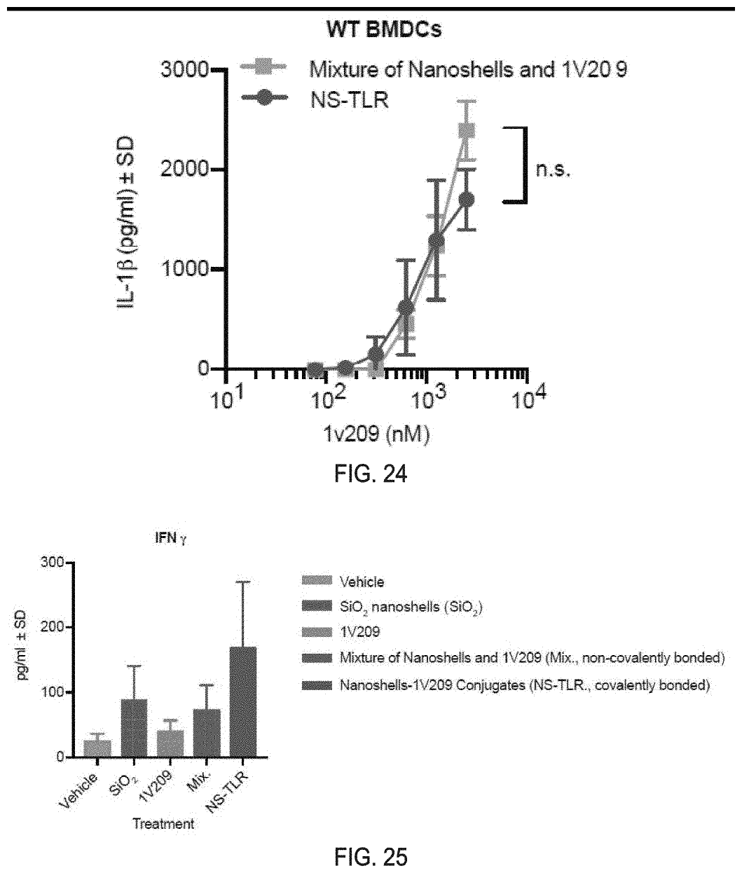

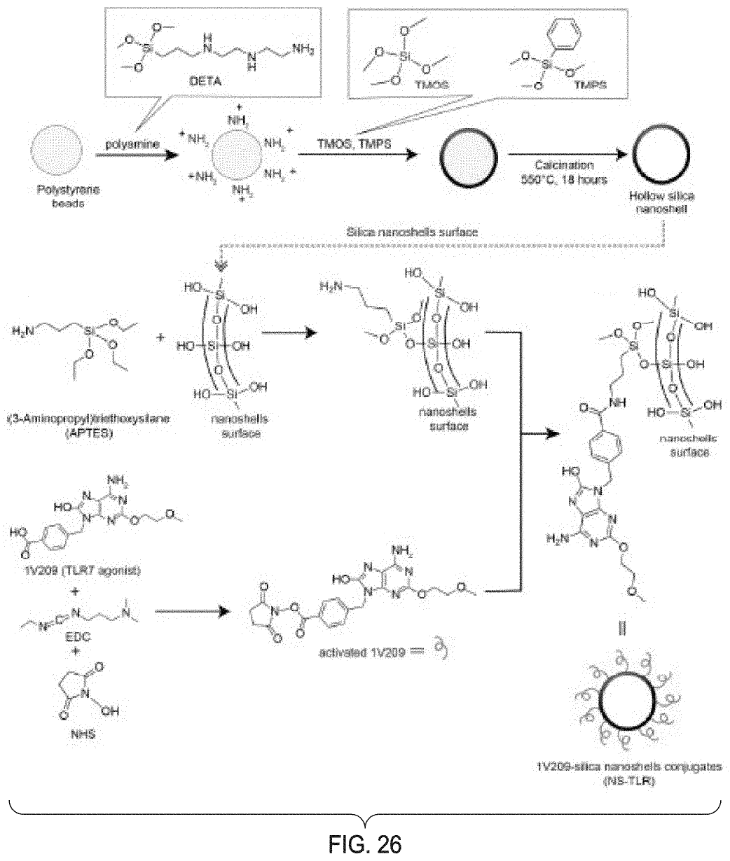

Having demonstrated that nanoparticles are locally retained at the injected site and being able to amplify immunity, we sought to investigate whether this NS-TLR7a has a safe profile for clinical use. Toxicology was evaluated for NS-TLR7a because a strong immunostimulatory agent (i.e., TLR7 agonists) usually results in undesired immunologic adverse effects. To analyze the hematological and biochemical effects by locally injected NS-TLR7a, 4-5 female BALB/c mice bearing CT26 tumors on right and left flanks, were i.t. injected 12.5 nmole of NS-TLR7a or vehicle (PBS) every other day for 6 treatments. The repeated treatments of NS-TLR7a did not induce mice body weight loss. On day 14, the blood and sera were collected for hematology and biochemistry analysis. As shown in FIGS. 16A, 17B and 19, there is no significant adverse effects on NS-TLR7a locally treated mice when compared to vehicle group. No abnormal behaviors were observed based on our animal clinic score observation that includes reduced activity, prone posture, difficulty in breathing, piloerection, lethargy, hunched posture, tachypnea, convulsion, tremors, ataxia, hairloss muzzle, scab on tail, matted fur, or dead immediately after injection or over the time course of treatment until day 14 post treatment. The only minor adverse events in the studies were limited to anemia (FIG. 16A). Anemia shown by reduced erythrocytes was observed following a TLR7 ligand treatment that was reported previously (Lanford et al., 2013) and the effects would attenuate throughout the study (Lanford et al., 2013), suggesting the reversibility of the effect. The immune cells including lymphocytes, monocytes, neutrophils, eosinophils, and basophils are not statistically different compared to the vehicle group. TLR7 is reported to induce the lymphopenia reaction that is mediated by type I IFN release by activated immune cells (Pockros et al., 2007; Baenziger et al., 2008). In our study, it is likely due to nanoshells localizing the TLR7 agonists to prevent such side effect toxicity. Similarly, at the given dose and administration routes, NS-TLR7a did not induce damage on hepatic, pancreatic, and renal function (FIG. 19). The electrolytes composition also remains stable after the treatment. This data suggested such nanoparticle-based TLR agonist (NS-TLR7a) is a safe agent that may be used in clinics.

TIL Analysis of Combination Therapy with NS-TLR7a, aPD1, and aCTLA4

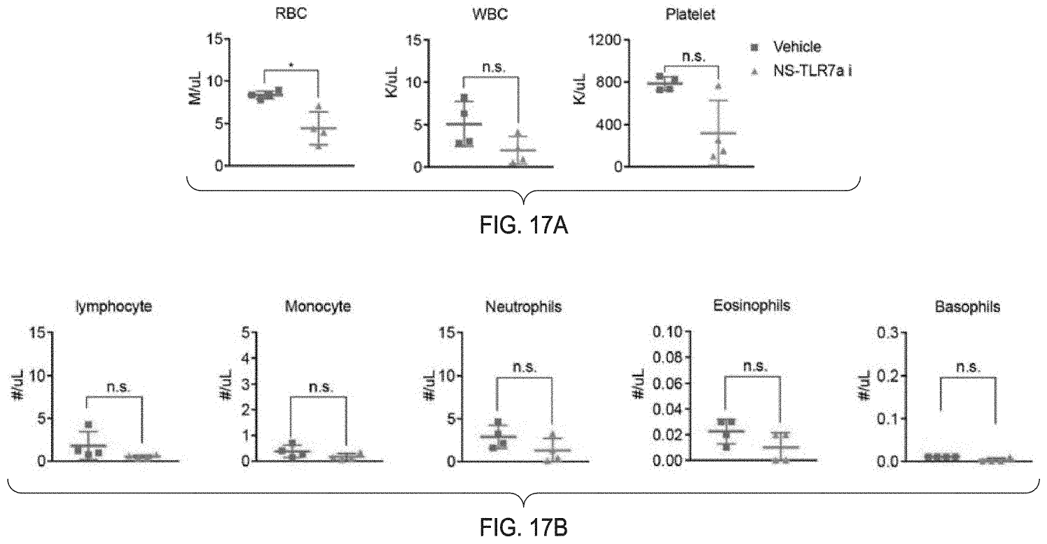

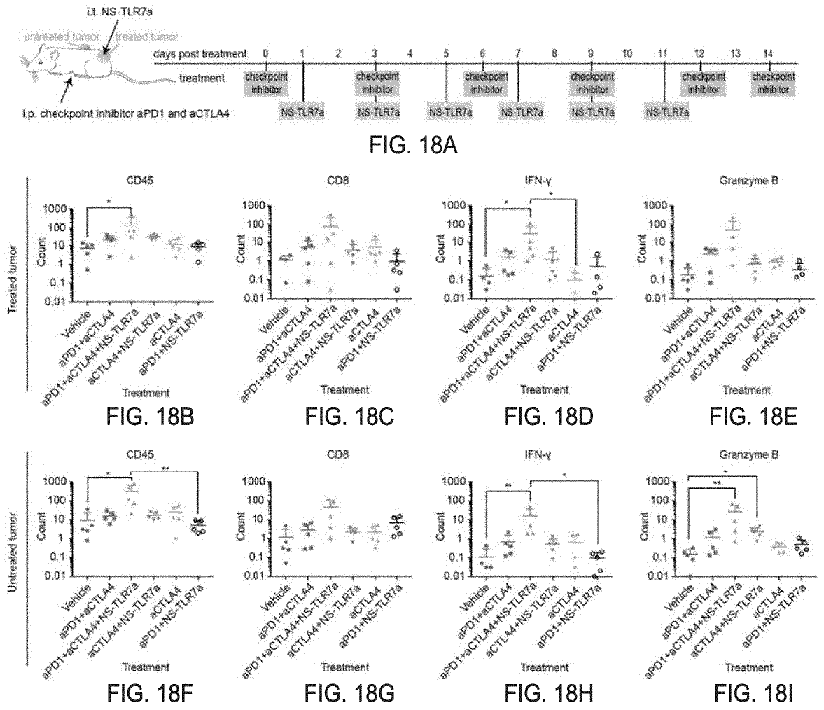

Tumors can create an immunosuppressive environment to promote tumor growth and metastasis (Rabinovich et al., 2007). TLR agonists, being a strong immunostimulatory agent, can reverse the immuno-suppressive tumor microenvironment (Sato-Kaneko et al., 2017). However, single agent of TLR7a did not result in tumor remission. Given the known efficacy of anti-PD-1 (aPD1) and anti-CTLA4 (aCTLA4) therapies and their ligand expression in this murine tumor type as well as human colorectal cancer, aPD1 and aCTLA4 therapy were employed and combined with NS-TLR7a. Next, to investigate the cell population in the colon tumor, we harvested both the treated and untreated distant tumor on 14 days post-treatment. Six treatment groups were used to study the cell population upon combination therapy: (1) vehicle, (2) aPD1+aCTLA4, (3) aPD1+aCTLA4+NS-TLR7a, (4) aCTLA4+NS-TLR7a, (5) aCTLA4, and (6) aPD1+NS-TLR7a. Single agent of aPD1 was not chosen as a group because previous studies have reported that single blockade of PD1 pathway has limited efficacy on CT26 tumors (Wang et al., 2016). The treatment protocol is shown in FIG. 17A that NS-TLR7a was injected i.t. and checkpoint inhibitor aPD1 and/or aCTLA4 were injected systemically. Both directly injected tumors (treated tumor) and contralateral not directly injected tumor (untreated tumors) were harvest on day 14 after first treatment to study the immune cells composition in the tumor environment. The NS-TLR7a+aPD1+aCTLA4 (triple therapy) treated group showed a higher CD45⁺ and CD8⁺ T cells (cytotoxic T cell) infiltration in both treated tumor and untreated tumor (FIGS. 15B, C, F, and G). The increase in IFN-γ and granzyme B in triple therapy also indicate those infiltrating T cells are at their active status (FIGS. 17 D, E, H, and I). The increase in both treated tumor and contralateral tumors demonstrate that the improvement of the immune response is systemic, regardless of only one site of the tumor being treated.

### NS-TLR7a Enhanced the Efficacy of Checkpoint Inhibitor and Improved Survival Rates

The therapeutic efficacy of NS-TLR7a was investigated. Since the combined checkpoint inhibitors (aPD1+aCTLA4) were reported to have promising therapeutic treatment outcome, it was chosen to compare with thriple therapy which consist of NS-TLR7a, aPD1, and aCTLA4. The dual block of PD1 and CTLA4 have shown ability to regress tumor growth on one tumor bearing mice, but treating on more than one tumor per mouse was less discussed (Duraiswamy et al, 2013). To know if NS-TLR7a can induce systemic and specific immune response to treat colon cancer, NS-TLR7a was combined together with aPD1 and aCTLA4 to treat two-colon tumor CT26-bearing mice. The treatment protocol is shown in FIG. 19A. As shown in FIGS. 16B-G, triple therapy that includes NS-TLR7a, aPD1 and aCTLA4 has induced tumors into full remission for both treated tumor site (full remission rate: 80%) and contralateral untreated tumor site (full remission rate: 60%). Based on our previous nanoparticle biodistribution study that the locally injected nanoparticles mainly stay at the injected site (FIG. 15), we believe that the tumor inhibition effects on contralateral tumor came from the systemic adaptive immune response but not the presence of NS-TLR7a that traveled to the distant tumor. The remission of the distant untreated tumor also suggested that the induced systemic immune response is tumor antigen specific. To validate that triple therapy induced tumor-specific adaptive immune responses, we monitored the growth of re-challenged tumors. For those mice who have induced tumor full remission, 10⁶ of CT26 cells were innoculated on non-treated flank site and back. None of the cured mice grow tumors (re-challenged mice tumor free rate: 100%), proving that the immune memory has been produced during the first time treatment. The therapeutic efficacy of NS-TLR7a with aPD1 and aCTLA4 has also been tested on mouse purchased from a different source (Envigo labotory) because it was reported that checkpoint inhibitor antitumor responsiveness varied by the source of the mice as tumor-bearing mice, possibly as a result of intestinal microbiome differences (data not shown). The mice purchased from Envigo labotory were the same subcutaneously injected 10⁶ of CT26 tumor cells on right and left flanks (n=5/group) and treated as the same protocol when tumors reach averagely 100 mm³, and then followed the same treatment protocol. Triple therapy on Envigo mice has induced 80% of full remission for both treated tumor site. The results showed that mice from different source demonstrate the similar therapeutic results that thriple combination of NS-TLR7a with aPD1 and aCTLA4 can induce full remission on both injected tumor and controlateral tumors. The treated tumor-free mice were also rechallenged with CT26 cells and none of mice re-grow tumors, which indicated the systemic and memorial immune response was established from the first combination treatment.

### CONCLUSION

In this study, the therapeutic efficacy of TLR7 agonists conjugated to silica nanoshells when used as monotherapy and as combination therapy with checkpoint inhibitors is described. Conjugating TLR7 ligands on to nanoshells lengthened the drug retention time at administration site and amplified immune response when compared to unconjugated counterparts. When combined with checkpoint inhibitors, locally injected TLR7 conjugates can induce a stronger immunity which is both systemic and tumor antigen-specific. The re-challenge studies of cured mice failed to re-grow tumor which indicated the cured mice has grown the long-term memory resistance to cancer cells. Against cancer types that are less responsive to checkpoint inhibitor treatments, potentially due to mismatch repair proficiency, conjugated TLR7 represents a reliable remedy to help induce a safe and more robost anti-tumor immune response.

TABLE 2
Comparison of combination cancer therapy with nanoparticles-based TLR7/8 agonists

| | Delivery platform | | | | | |
|---|---|---|---|---|---|---|
| | | Size | TLR7/8 agonists | | Combination | |
| Name | Material | (nm) | Agent | Inject | therapy | effect |
| NS-TLR7a | silica hollow | 100 | 1V209 | i.t. | aPD1, | *Conjugation improve T |
| | shells | | | | aCTLA4 | cell infiltration into tumor |
| | | | | | | compared to unconjugated |
| | | | | | | TLR7 agonists *Improved |
| | | | | | | systemic cytokine *Stayed |
| | | | | | | at administration site after |
| | | | | | | injection |
| | | | | | | *Showed a safe usage |
| | | | | | | profile after multiple |
| | | | | | | dosages |
| | | | | | | *Induced neoantigen- |
| | | | | | | specific systemic immune |
| | | | | | | response |
| | | | | | | *Had ability to induce |
| | | | | | | colorectal tumors into |
| | | | | | | remission and establish a |
| | | | | | | resistant immune memory |
| CDNP-R848 | β-cyclodextrin | 30 | R848 | i.v. | aPD1 | *Delivered drug to tumor- |
| | (CD) | | | | | associated macrophages |
| | | | | | | and direct to M1 phenotype |
| | | | | | | *Slowed down tumor |
| | | | | | | growth |
| | | | | | | *Improved response rate |
| | | | | | | when combined with aPD1 |
| IMDQ^{nano} | block co- | 50 | imidazoquinoline | p.t. | PDL1 | *Activated DC in sentinel |
| | polymer | | | | nanobodies | lymph nodes |
| | (mTEGMA | | | | and FLT3L | *Improved proliferation of |
| | and PFPMA) | | | | | antigen-specific CDS T |
| | | | | | | cells |
| | | | | | | *Slowed down tumor |
| | | | | | | growth when combined |
| | | | | | | with aPDL1 nanobodies |
| | | | | | | and FLT3L |
| 522GGNP | acid- | 200 | 522 | s.c. | OVA | *Activated DC and CD8 T |
| | responsive | | | | | cell. induce stronger NK |
| | PLGA NPs | | | | | cell responses than |
| | | | | | | conventional PLGA NP. |
| | | | | | | *Slowed down tumor |
| | | | | | | growth |
| PLGA-ICG- | PLGA | 100 | R837 | s.c, i.t. | aCTLA4, | *Increased CD8 CTL in |
| R837 | (encapsulated | | | | PTT | secondary tumor |
| | ICG) | | | | | *Slowed down tumor |
| | | | | | | growth of 2nd tumor |
| PLGA-PEG- | PEG grafted | 100 | R837 | i.v. | aCTLA4, | *Accumulated in tumors |
| ICG-R837 NPs | PLGA co- | | | | PTT | *Induce higher DC |
| | polymer | | | | | maturation and enhanced |
| | | | | | | pro-inflammatory cytokines |
| | | | | | | in serum |
| | | | | | | *Delayed secondary tumor |
| HA- | PHIS and | 200 | R848 | i.v. | DOX | *Better control tumor |
| DOX/PHIS/ | HA | | | | | growth compared to free |
| R848 NPs | | | | | | DOX, free R848 or mixture |
| | | | | | | of DOX/R848 |
| | | | | | | *Lung metastasis was |
| | | | | | | almost unseen |
| | | | | | | *Increased tumor-infiltrating T |
| | | | | | | CD3 and CD8 cells |
| PD-1-targeted | PLGA based | 267- | R848 | i.v. | anti-PD-1, | *Increased CD8 T cells in |
| R848 NP | NPs | 269 | | | | MC38 and B16 tumors |
| | | | | | | *Delayed tumor growth |
| | | | | | | and improved survival |
| | | | | | | *Sensitized tumors to anti- |
| | | | | | | PD-1 antibody responsive |
| Gardi-PLGA | PLGA NPs | 194 | gardiquimod | i.t. | DMXAA | *Inhibited tumor growth |
| | | | | | | and improve survival |

NPs = nanoparticles,
mTEGMA = methoxytriethyleneglycol methacrylate,
PFPMA = pentafluorophenyl methacrylate;
PLGA = Poly(lactic-co-glycolic acid);
PEG = olyethylene glycol;
ICG = Indocyanine green;
PHIS = poly(L-histidine);
HA = hyaluronic acid;
R848 = resiquimod;
R837 = imiquimod;
i.t. = intratumoral;
i.v. = intravemous;
s.c. = subcutaneous;
p.t. = peritumoral;
OVA = Ovalbumin;
PTT = photothermal therapy;
DOX = Dorubicin (a chemodrug);
DMXAA = 5,6-Dimethylxanthenone-4-acetic acid (a vasculature disrupting agent)

TABLE 3
1V209 ligand quantification of NS-TLR

| | Ligand dependency | | | Size dependency | | |
|---|---|---|---|---|---|---|
| Diameter (nm) | 100 | 100 | 100 | 100 | 500 | 2,000 |
| Formula | low | medium | high | high | high | high |
| | density | density | density | density | density | density |
| NS mass ^{a} (g) | 5.82E−16 | 5.82E−16 | 5.82E−16 | 5.82E−16 | 1.72E−14 | 2.84E−13 |
| NS/mg | 1.72E12 | 1.72E12 | 1.72E12 | 1.72E12 | 5.82E10 | 3.53E9 |
| Ligands/NS ^{b} | 1,000 | 1,000-6,000 | >6,000 | ~6,000 | ~160,000 | −2,750,000 |
| Area (nm²)/NS | 31,400 | 31,400 | 31,400 | 31,400 | 785,000 | 1,2560,000 |
| Ligands/nm² | <0.03 | 0.03-0.20 | >0.20 | ~0.20 | ~0.20 | ~0.20 |

^{a} Nanoshell mass was calculated by assuming silica density = 2.28 g/cm³. ¹
^{b} Ligands were quantified based on previous calculation.¹

TABLE 4
Drug potency of NS-TLR and unconjugated 1V209

| | Unconjugated | | Conjugated ligand density | | |
|---|---|---|---|---|---|
| Potency | Silica Nanoshells | 1V209 | low | medium | high |
| EC_{50, IL-6}^{a} | u.d.l.^{b} | 1075 ± 101 | n.d.^{c} | 756 ± 224 | 279 ± 118 |
| E_{max, IL-6}^{a} | u.d.l.^{b} | 9.9 ± 1.0 (1×) | 1.4 ± 0.3 (0.1×) | 14.8 ± 1.8 (1.5×) | 14.8 ± 1.5 (1.5×) |
| EC_{50, IL-12}^{a} | u.d.l.^{b} | 883 ± 193 | n.d.^{c} | 389 ± 84 | 262 ± 20 |
| E_{max, IL-12}^{a} | u.d.l.^{b} | 3.2 ± 2.3 (1×) | 0.6 ±0.3 (0.2×) | 7.2 ± 4.8 (2.2×) | 93 ± 6.6 (2.9×) |

^{a}EC₅₀ (nM) and Eₘₐₓ (ng/mL) was calculated using Prism software. Data shown are means + SD. Emax was normalized to unconjugated 1V209 (=1X)
^{b}u.d.l.; under the detection limits
^{c}n.d.; not determined

| Nanoparticle type | size (nm) | Surface Compound | Payload | IL-1β secretion | Immune Effect |
|---|---|---|---|---|---|
| Hollow silica shells | 100 | TLR7 agonists | OVA | Yes | Improved TLR7 agonistic |
| | | | | | activity (increased |
| | | | | | IL-6, IL-12 cytokines |
| | | | | | production) |
| | | | | | Amplified Th1 and Th2 |
| | | | | | immunity - Stimulated |
| | | | | | IL-1β secretion |
| Mesoporous silica | 88,000 × | Naked | GM-CSF, CpG, OVA | n.d.^{a} | Recruited DC Enhanced |
| rods | 4,500 | | | | Th1 and Th2 and cytotoxic |
| | | | | | T cells |
| Fibrous mesoporous | 100-200 | Naked | OVA, poly IC | n.d.^{a} | Improved maturation of bone |
| silica | | | | | marrow derived dendritic cells |
| | | | | | in vitro |
| Ca, Mg, Zn-doped | 100 | Naked | OVA | n.d.^{a} | Zn doped particles induced |
| | | | | | higher Th1 immunity |
| Dendritic mesoporous | 350 | Naked | tumor fragment s, | n.d.^{a} | Improved anti-tumor activity |
| organosilica | | | OVA | | Increased IFN-γ, IL-12 and |
| Mesoporous silica | 50-200 × | Naked | OVA | n.d.^{a} | Naked and NH₂ |
| nanorods | 10-30 | | | | modified MSNRs |
| | | | | | exhibited Th2-skewed |
| | | | | | immunity |
| Hollow Mesoporous | 200 | Naked | OVA and poly | n.d.^{a} | Improved anti-tumor |
| | | | | | ability Promoted |
| | | | | | T cell population |
| Hollow Mesoporous | 200 | Naked | — | n.d.^{a} | Promoted LPS uptake |
| silica nanospheres | | | | | Induced anti-tumor activity |
| | | | | | with tumor fragments |
| | | | | | Increased memory T cell |
| S1 | 430 | Naked | BSA | n.d.^{a} | Increased IgG and IgA titers. |
| S2 | 130 | Naked | BSA | n.d.^{a} | Induced higher level of IgG |
| | | | | | and IgA titer than SBA-15 |
| SBA-15 | 2 μm long | Naked | BSA molecules | n.d.^{a} | Improved the immune response |
| | | | | | of low responder |

^{a}not discussed in the studies

### REFERENCES

Al-Abd et al., J. Control. Release, 142:101 (2010).
Arora & Mahalingam, J. Gastrointest. Oncol., 9:170 (2018).
Bachmann et al., Nat. Rev. Immunol., 10:787 (2010).
Baenziger et al., Blood, 13:377 (2008).
Boyiadzis et al., J. Immunother. cancer, 6:35 (2018).
Brown et al., Biodistribution and Toxicity of Micellar Platinum Nanoparticles in Mice via Intravenous Administration. Nanomater. (Basel, Switzerland) 8, (2018).
Cassidy & Syed, Nat. Rev. Drug Discov., 16:525 (2017).
Castle et al., BMC Genomics, 15:190 (2014).
Chan et al., Bioconjug. Chem., 20:1194 (2009).
Chen et al., Nat. Commun., 7:13193 (2016).
Ciombor et al., Annu. Rev. Med. 6683 (2015).
Cogdill et al., Br. J. Cancer, 117:1 (2017).
Dockrell, J. Antimicrob. Chemother., 48:751 (2001).
Duan & Li, Small, 9:1521 (2013).
Duraiswamy et al., Cancer Res., 73:3591 (2013).
Efremova et al., Nat. Commun., 2:32 (2018).
Emambux et al., Expert Opin. Biol. Ther., 18:561 (2018).
Engel et al., Expert Rev. Clin. Pharmacol., 4:275 (2011).
Fakih, J. Clin. Oncol., 33; 1809 (2015).
Fellner, Promising Drugs in Clinical Development To Treat Advanced Colorectal Cancer. P T 42, 262-265 (2017).
Friedman et al., Curr. Pharm. Des., 19:6315 (2013).
Ghiringhelli et al., Nat. Med., 15:1170 (2009).
Gómez et al., Innate Immun., 23:697 (2017).
He et al., Trends Biochem. Sci., 41:1012 (2016).
Hodi et al., Lancet Oncol., 17:1558 (2016).
Ka et al., Front. Immunol., 5:400 (2014).
Kawai et al., Trends Mol. Med., 13:460 (2007).
Kim et al., Bioconjug. Chem., 27:2007 (2016).
Kim et al., Cancer Immunol. Res., 2:91 (2014).
Kim et al., Nanoscale, 10:20851 (2018).
Knutson et al., Cancer Immunol. Immunother., 5:721 (2005).
Kusaka et al., PLoS One, 9:e92634 (2014).
Lanford et al., Gastroenterology, 144:1508 (2013).
Le et al., J. Clin. Oncol., 34:103 (2016).
Le et al., N. Engl. J. Med., 372:2509 (2015).
Le et al., Science (New York, N.Y.) 357, (American Association for the Advancement of Science, 2017).
Liu et al., Acta Biomater., 66:310 (2018).
Livak & Schmittgen, Methods, 25:402 (2001).
Mark et al., J. Infect. Dis., 195:1324 (2007).
Meyer et al., Expert Opin. Investig. Drugs, 22:149 (2013).
Milling et al., Adv. Drug Deliv. Rev., 114:79 (2017).
Morishige et al., Biomaterials 31:6833 (2010).
Nuhn et al., pH-degradable imidazoquinoline-ligated nanogels for lymph node-focused immune activation. doi:10.1073/pnas.1600816113
Nuhn, et al., Adv. Mater., 30:1803397 (2018).
Overman et al., Lancet Oncol., 18:1182 (2017).
Pockros et al., J. Hepatol., 47:174 (2007).
Rabinovich et al., Annu. Rev. Immunol., 25:267 (2007).
Rodell et al., Nat. Biomed. Eng., 2:578 (2018).
Sandoval et al., J. Biomed. Opt., 20:88003 (2015).
Sato-Kaneko, JCI Insight 2, (2017).
Schmid et al., Nat. Commun., 8:1747 (2017).
Scho et al., Oncogene, 27:190 (2008).
Seth et al., Oncotarget, 8:5371 (2017).
Shinchi et al., Bioconjug. Chem., 26:1713 (2015).
Soria et al., DOI Antimicrob. Agents Chemother., 4712: (2003).
Sun et al., World J. Gastroenterol., 22:6362 (2016).
Tosolini et al., Cancer Res., 71:1263 (2011).
Wang et al., Proc. Natl. Acad. Sci. U.S.A., 113:E7240 (2016).
Yang et al., Sci. Rep., 7:3303 (2017).
Yu et al., _, 5:5717 (2011).
Zanetti, J. Immunol., 194:2049 (2015).
Zhang et al., J. Am. Chem. Soc. 134:15790 (2012).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A composition comprising a silica shell conjugated to a TLR7 agonist.

2. The composition of claim 1 wherein the shell has a diameter less than about 200 nm.

3. The composition of claim 1 wherein the TLR7 agonist comprises formula (II): 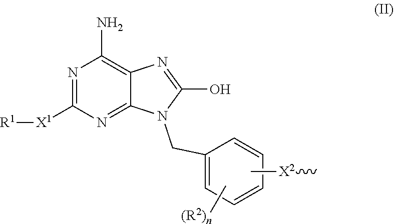
wherein X₁ is —O—, —S—, or —NR^{c}—;
R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;
R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;
each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;
each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;
wherein the substituents on any alkyl, aryl or heterocyclic groups are hydroxy, C₁₋₆alkyl, hydroxyC₁₋₆alkylene, C₁₋₆alkoxy, C₃₋₆cycloalkyl, C₁₋₆alkoxyC₁₋₆alkylene, amino, cyano, halo, or aryl;
n is 0, 1, 2, 3 or 4; and
X² is absent, (C₁-C₁₀)alkyl, substituted (C₁-C₁₀)alkyl, or is a bond or a linking group;
or a tautomer thereof;
or a pharmaceutically acceptable salt or solvate thereof.

4. The composition of claim 1 wherein the TLR7 agonist comprises 1V209, imiquimod, resiquimod, or SM360320.

5. The composition of claim 1 further comprising a checkpoint inhibitor.

6. The composition of claim 5 wherein the inhibitor comprises pembrolizumab, nivolumab, atezolizumab, avelumab, durvalumab, or ipilimumab.

7. The composition of claim 3 wherein X² comprises N-hydroxysuccinamide.

8. The composition of claim 3 wherein prior to conjugation the silica shell comprises an amino propyl triethoxysilane.

9. The composition of claim 3 wherein each R2 independently comprises (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, substituted aryl(C₁-C₆)alkyl, alkoxy substituted C6 aryl, Het, Het (C₁-C₆)alkyl, or (C₁-C₆)alkoxycarbonyl or each R2 independently comprises H, —C₁-C₆ alkyl, —C₁-C₆ alkoxy, —NR^{a}R^{b}, —N₃, —OH, —CN, —COOH, —COOR¹, —C₁-C₆ alkyl-NR^{a}R^{b}, C₁-C₆ alkyl-OH, C₁-C₆ alkyl-CN, C₁-C₆ alkyl-COOH, C₁-C₆ alkyl-COOR¹, 5-6 membered ring, substituted 5-6 membered ring, —C₁-C₆ alkyl-5-6 membered ring, —C₁-C₆ alkyl-substituted 5-6 membered ring C₂-C₉ heterocyclic, or substituted C₂-C₉ heterocyclic.

10. The composition of claim 3 wherein R¹ comprises substituted (C₁-C₁₀)alkyl.

11. A method to inhibit or treat cancer, comprising administering to a mammal in need thereof an effective amount of the composition of claim 1, optionally in conjunction with one or more chemotherapeutics or anti-cancer antibodies.

12. The method of claim 11 wherein the mammal is a human.

13. The method of claim 11 further comprising administering a checkpoint inhibitor.

14. The method of claim 13 wherein the inhibitor comprises pembrolizumab, nivolumab, atezolizumab, avelumab, durvalumab, or ipilimumab.

15. The method of claim 11 wherein the composition is parenterally administered.

16. The method of claim 11 wherein the composition is intravenously administered.

17. The method of claim 11 wherein the composition is locally administered.

18. The method of claim 11 wherein the composition is intratumorally administered.

19. The method of claim 18 further comprising employing ultrasound guided histotripsy.

20. The method of claim 11 wherein the cancer is bladder cancer, prostate cancer, testicular cancer, lung cancer, breast cancer, brain cancer, liver cancer, ovarian cancer, kidney cancer, or pancreatic cancer.


SEQUENCE LISTING (6 sequences)

SEQ ID NO: 1 - DNA, 20 bp, Artificial Sequence
  A synthetic oligonucleotide
tcagcagcgactccttttcc

SEQ ID NO: 2 - DNA, 20 bp, Artificial Sequence
  A synthetic oligonucleotide
catggcctcagttccgaaaa

SEQ ID NO: 3 - DNA, 20 bp, Artificial Sequence
  A synthetic oligonucleotide
tagggcctctcttgctcagt

SEQ ID NO: 4 - DNA, 20 bp, Artificial Sequence
  A synthetic oligonucleotide
ttcttcagcaacagcaaggc

SEQ ID NO: 5 - DNA, 17 bp, Artificial Sequence
  A synthetic oligonucleotide
cgagccgcctggatacc

SEQ ID NO: 6 - DNA, 24 bp, Artificial Sequence
  A synthetic oligonucleotide
tcaagctcatttcctggtatgaca